(19) United States
(12) Patent Application Publication
(10) Pub. No.: US 2022/0347144 A1
(43) Pub. Date: Nov. 3, 2022

(54) COMPOSITIONS AND METHODS FOR PREVENTING AND/OR REDUCING WEIGHT GAIN AND ASSOCIATED CONDITIONS

(71) Applicant: University of Virginia Patent Foundation, Charlottesville, VA (US)

(72) Inventors: Todd E. Fox, Charlottesville (US); Mark Kester, Afton (US)

(73) Assignee: University of Virginia Patent Foundation, Charlottesville, VA (US)

(21) Appl. No.: 17/763,798

(22) PCT Filed: Sep. 28, 2020
(86) PCT No.: PCT/US2020/053117

§ 371 Date: Mar. 25, 2022

Related U.S. Application Data
(60) Provisional application No. 62/906,854, filed on Sep. 27, 2019.

(51) Int. Cl. A61K 31/201; A61P 3/04; A61P 3/06; A61P 3/10; A61K 31/231

(52) U.S. Cl. CPC A61K 31/201; A61P 3/04; A61P 3/06; A61P 3/10; A61K 31/231

(57) ABSTRACT

Provided are methods for preventing and/or reducing weight gain in subjects. In some embodiments, the methods include administering an effective amount of a composition that includes a long chain fatty acid of at least 22 carbons and/or a derivative thereof to prevent and/or reduce weight gain in the subject. In some embodiments, the long chain fatty acid is a monounsaturated omega 9 fatty acid, which in some embodiments is nervonic acid or a derivative thereof. Also provided are methods for preventing and/or reducing the development of obesity in subjects, for inhibiting reduction of very-long chain sphingolipids in subjects, for increasing content of one or more ceramides while simultaneously decreasing content of one or more C20-C26 ceramides in subjects, and for reducing blood glucose levels resulting from consumption of high fat diets in subjects.

### CROSS REFERENCE TO RELATED APPLICATION

The presently disclosed subject matter claims the benefit of U.S. Provisional Patent Application Ser. No. 62/906,854, filed Sep. 27, 2019, the disclosure of which incorporated herein by reference in its entirety.

### GOVERNMENT INTEREST

This invention was made with government support under Grant No. EY018336 awarded by The National Institutes of Health. The government has certain rights in the invention.

### TECHNICAL FIELD

The presently disclosed subject matter relates in some embodiments to methods and compositions for preventing and/or reducing weight gain in subjects. In some embodiments, the presently disclosed subject matter relates to compositions and methods for preventing and/or reducing the development of obesity and/or for inhibiting reduction of very-long chain sphingolipids in a mammal. More particularly, the presently disclosed subject matter relates to administering an effective amount of a long chain fatty acid, in some embodiments a long chain fatty acid of at least 22 carbons, a precursor thereof, a metabolite thereof, an analog thereof, or any combination thereof to a mammal in order to prevent and/or reduce diet-related weight gain and/or inhibit reduction of very-long chain sphingolipids in a mammal. In some embodiments, the long chain fatty acid is an omega 9 monounsaturated fatty acid, which in some embodiments includes erucic acid, nervonic acid, and/or ximenic acid.

### BACKGROUND

Alterations in specific fatty acid constituents within specific lipid class continues to gain greater appreciation in both normal homeostasis and in pathophysiology including obesity, metabolic syndrome, and diabetes (Hammad & Jones, 2017; Palomer et al., 2018; Bandet et al., 2019). Obese individuals have excess circulating free fatty acids (FFAs) and altered lipid composition within most tissues. These alterations have pathological consequences by contributing to insulin resistance, inflammation, and lipotoxicity. One particular lipid class of interest are sphingolipids, which have been implicated in contributing to metabolic dysfunction and insulin resistance (Fox & Kester, 2010; Meikle & Summers, 2017; Lambert et al., 2018; Chaurasia et al., 2019).

A major type of sphingolipids are ceramides. Ceramides, are comprised of a sphingoid backbone coupled to a fatty acyl-chain of different lengths through an amide linkage. A family of six known ceramide synthases (CerS) catalyze the fatty acid acylation step, and these isoforms differ in tissue distribution and fatty acyl-CoA specificity (Park et al., 2014). Emerging evidence implicate that the acyl-ceramide composition influences biological responses (Park et al., 2014). Animal knockouts of specific CerS, and consequently changes in ceramide and sphingolipid fatty acid composition, leads to changed disease phenotype depending on the biological context. For obesity, knockouts of the C16 (palmitate)-ceramide generating CerS5 (Gosejacob et al., 2016) or CerS6 (Turpin et al., 2014) leads to protection against diet-induced obesity and resulting phenotype. Furthermore, mouse haplotypes of the predominantly C22-C24-ceramide generating CerS2, leads to increased C16-ceramides and consequently more susceptibility to diet-induced steatohepatitis and insulin resistance (Raichur et al., 2014).

It has been previously demonstrated that C24:1(nervonate)-ceramides, a predominant ceramide within liver tissue is reduced in various models of type 1 diabetes and within a diet-induced mouse model of obesity (Fox et al., 2011). Though the influence of elevated C16-ceramides have received more study, the role of reduced very long-chain ceramides, such as C24:1-ceramide, are largely unknown. A few human-based studies have supported our preclinical models for reduced nervonic acid in obesity and metabolic syndrome. A negative correlation between nervonic acid and obesity-related risk factors has been observed (Oda et al., 2005). Plasma nervonic acid was significantly lower in obese compared to lean participants and was inversely correlated with BMI (Pickens et al., 2015). Furthermore, a study on Japanese males found that subjects with metabolic syndrome demonstrated reduced nervonic acid in serum lipids compared to subjects without metabolic syndrome (Yamazaki et al., 2014). To extend beyond these correlative studies, herein are described metabolic studies investigating restoring nervonic acid via the diet and the effect on obesity and related metabolic complications.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates in some embodiments to methods for preventing and/or reducing weight gain in subjections, optionally mammals, further optionally humans. In some embodiments, the methods comprise, consist essentially of, or consist of administering to the subject an effective to amount of a composition comprising, consisting essentially of, or consisting of long chain fatty acid of at least 22 carbons and/or a derivative thereof, optionally wherein the long chain fatty acid is a monounsaturated omega 9 fatty acid, wherein the effective amount of the composition is effective for preventing or reducing weight gain in the mammal relative to that which would have occurred in the mammal in the absence of the composition.

In some embodiments, the presently disclosed subject matter also relates in some embodiments to methods for preventing or reducing the development of obesity in subjects, optionally mammals, further optionally humans. In some embodiments, the methods comprise, consist essentially of, or consist of administering to the subject an effective amount of a comprising, consisting essentially of, or consisting of long chain fatty acid of at least 22 carbons and/or a derivative thereof, optionally wherein the long chain fatty acid is a monounsaturated omega 9 fatty acid.

In some embodiments of the presently disclosed methods, the long chain fatty acid is selected from the group consisting of erucic acid, nervonic acid, and ximenic acid. In some embodiments, the derivative of the long chain fatty acid of at least 22 carbons is a precursor thereof, a metabolite thereof, an analog thereof, an ester thereof, a pharmaceutically acceptable salt thereof, or any combination thereof. In some embodiments, the derivative comprises the long chain fatty acid bioconjugated to a biomolecule selected from the group consisting of a ceramide, a lipid, a phospholipid, a cholesterol, a diglyceride, a triglyceride, a monoacylglycerol, and a glycerophospholipid. In some embodiments, the long chain fatty acid if bioconjugated to the biomolecule via an ester linkage, an ether linkage, an amide linkage, or any combination thereof. In some embodiments, the derivative is an ester of the long chain fatty acid and a C₁-C₆ straight chain biomolecule, a C₁-C₆ branched chain biomolecule, or is any combination thereof. In some embodiments, the derivative of the long chain fatty acid is a methyl ester, an ethyl ester, or any combination thereof.

In some embodiments of the presently disclosed methods, the composition comprises, consists essentially of, or consists of nervonic acid, optionally an ester thereof, and further optionally a methyl ester and/or an ethyl ester thereof.

In some embodiments of the presently disclosed methods, the composition comprises, consists essentially of, or consists of nervonic acid and/or a derivative thereof bioconjugated to a ceramide, optionally wherein the derivative thereof is a nervonic acid ethyl ester.

In some embodiments, the subject is a human.

In some embodiments, the presently disclosed subject matter relates in some embodiments to methods for inhibiting reduction of very-long chain sphingolipids in subjects, optionally mammals, further optionally humans. In some embodiments, the methods comprise, consist essentially of, or consist of administering to the subject an effective amount of a composition comprising, consisting essentially of, or consisting of long chain fatty acid of at least 22 carbons, a precursor thereof, a metabolite thereof, an analog thereof, an ester thereof, a pharmaceutically acceptable salt thereof, or any combination thereof, optionally wherein the long chain fatty acid is a monounsaturated omega 9 fatty acid. In some embodiments, the long chain fatty acid is C24:1 nervonic acid or a derivative thereof. In some embodiments, the long chain fatty acid is selected from the group consisting of erucic acid, nervonic acid, and ximenic acid. In some embodiments, the ester thereof is a C₁-C₆ straight chain ester, C₁-C₆ branched chain ester, or any combination thereof. In some embodiments, the ester thereof is a methyl ester, an ethyl ester, or any combination thereof.

In some embodiments of the presently disclosed methods, the composition comprises, consists essentially of, or consists of nervonic acid, optionally an ester thereof, and further optionally a methyl ester and/or an ethyl ester thereof.

In some embodiments of the presently disclosed methods, the weight gain, the obesity, or the reduction in very-long chain sphingolipids is a consequence of the mammal consuming a high fat diet.

In some embodiments, the subject is a human.

The presently disclosed subject matter also relates in some embodiments to method for increasing content of one or more first species of ceramides in a mammal while simultaneously decreasing content of one or more second species of C20-C26 ceramides in the mammal, the method comprising, consisting essentially of, or consisting of administering to the mammal an effective amount of a composition comprising, consisting essentially of, or consisting of an effective amount of a composition comprising, consisting essentially of, or consisting of long chain fatty acid of at least 22 carbons, a precursor thereof, a metabolite thereof, an analog thereof, an ester thereof, a pharmaceutically acceptable salt thereof, or any combination thereof, optionally wherein the long chain fatty acid is a monounsaturated omega 9 fatty acid, and further wherein the effective amount is effective for increasing content of the one or more first species of ceramides in the mammal while simultaneously decreasing content of the one or more second species of C20-C26 ceramides in the mammal. In some embodiments, the one or more first species of ceramides increased are selected from the group consisting of C24:1-lysophosphatidylcholine an diacylglycerides C16:0/C24:1, C18:0/C24:1, C18:1/C24:1, and C18:2/C24:1. In some embodiments, the content is plasma content, liver content, or both.

The presently disclosed subject matter also relates in some embodiments to methods for reducing blood glucose levels resulting from consumption of a high fat diet in subjects in need thereof. In some embodiments, the methods comprise, consist essentially of, or consist of administering to the subject an effective amount of a composition comprising, consisting essentially of, or consisting of an effective amount of a composition comprising, consisting essentially of, or consisting of long chain fatty acid of at least 22 carbons and/or a derivative thereof, optionally a precursor thereof, a metabolite thereof, an analog thereof, an ester thereof, a pharmaceutically acceptable salt thereof, or any combination thereof, further optionally wherein the long chain fatty acid is a monounsaturated omega 9 fatty acid, wherein the effective amount is effective for reducing the blood glucose level in the subject. In some embodiments, the long chain fatty acid is selected from the group consisting of erucic acid, nervonic acid, and ximenic acid. In some embodiments, the ester thereof is a C₁-C₆ straight chain ester, C₁-C₆ branched chain ester, or any combination thereof. In some embodiments, the ester thereof is a methyl ester, an ethyl ester, or any combination thereof.

In some embodiments of the presently disclosed methods, the composition comprises, consists essentially of, or consists of nervonic acid, optionally an ester thereof, and further optionally a methyl ester and/or an ethyl ester thereof. In some embodiments, the long chain fatty acid is nervonic acid and the nervonic acid is selected from the group consisting of free nervonic acid, nervonic acid methyl ester, nervonic acid ethyl ester, and nervonic acid bioconjugated to a ceramide, a phospholipid, a cholesterol, a diglyceride, a triglyceride, a sphingolipid, a monoacyl glycerol, a glycerophospholipid, or any combination thereof.

In some embodiments of the presently disclosed methods, the composition is administered as part of a nanoscale or microscale delivery vehicle, wherein the delivery vehicle is optionally selected from the group consisting of a liposome, a lipo/polymer, a microparticle, and a nanoparticle, or any combination thereof. In some embodiments, the delivery vehicle comprises a nanoliposome, and further wherein the nanoliposome encompasses the long chain fatty acid, the precursor thereof, the metabolite thereof, the analog thereof, the ester thereof, the pharmaceutically acceptable salt thereof, the combination thereof and/or comprises a lipid bilayer that comprises the long chain fatty acid, the precursor thereof, the metabolite thereof, the analog thereof, the ester thereof, the pharmaceutically acceptable salt thereof, the combination thereof. In some embodiments, the delivery vehicle is biodegradable in a cell, tissue, organ, or fluid of a subject. In some embodiments, the delivery vehicle is designed to biodegrade in the subject in order to release the long chain fatty acid of at least 22 carbons, the precursor thereof, the metabolite thereof, the analog thereof, the ester thereof, the pharmaceutically acceptable salt thereof, or any combination thereof to the subject over a period of time. In some embodiments, the delivery vehicle releases the long chain fatty acid of at least 22 carbons, the precursor thereof, the metabolite thereof, the analog thereof, the ester thereof, the pharmaceutically acceptable salt thereof, or any combination thereof to the subject's circulation and/or a cell, tissue, and/or organ of subject over the period of time. In some embodiments, the delivery vehicle is designed to biodegrade subsequent to contact with the subject's digestive system or circulatory system. In some embodiments, the delivery vehicle is designed to degrade in the subject to release at least about 50% of the long chain fatty acid of at least 22 carbons, the precursor thereof, the metabolite thereof, the analog thereof, the ester thereof, the pharmaceutically acceptable salt thereof, or any combination thereof over a period of time of at least 30 minutes, at least 1 hour, at least 6 hours, at least 12 hours, at least 24 hours, or longer than 24 hours.

Accordingly, it is an object of the presently disclosed subject matter to provide methods and compositions for preventing and/or reducing weight gain in subjects in need thereof in order to improve physiological outcomes in the subjects.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Detailed Description, Figures, and EXAMPLES.

### BRIEF DESCRIPTIONS OF THE DRAWINGS

FIGS. 1A-1J: Ceramides are altered in models of diet-induced obesity.

FIG. 1A: Ceramide composition was assessed by LC-MS/MS of liver samples derived from male C57B16/J mice (n=10/group) fed a control or 60% high fat chow, or isocaloric diets supplemented with nervonic acid (0.6%). The sum of the reported ceramide species is shown in the inset. Other NA-containing lipids were also quantified, including C24:1-hexosylceramide (FIG. 1B), C24:1 sphingomyelin (FIG. 1C), C24:1-lysophosphatidylcholine (LPC; FIG. 1D), and the diacylglycerides (DG) C16:0/C24:1 (FIG. 1E), C18:0/C24:1 (FIG. 1F), C18:1/C24:1 (FIG. 1G), and C18:2/C24:1 (FIG. 1H). Figure if Plasma C24:1-ceramides were assessed after 72 hours on the indicated diets (n=4/group). For FIGS. 1A-1I, 2-way ANOVA did not reveal significant interactions between the diets and NA. FIG. 1K: CerS2 knockdown decreased C24:1 ceramide, which is only partially restored by 1 μM NA treatment for 24 hours in HEK293 cells (n=3), CerS2 siRNA diminished mRNA by 70% (not shown). FIG. 1J: Ceramide composition was determined from male Sprague-Dawley rats (n=5/group) fed a low fat, a 40%, or a 60% fat containing diet. One-way ANOVA was utilized to assess differences between groups (* p<0.05, ** p<0.01, *** p<0.001).

FIGS. 2A-2D: Nervonic acid prevents diet-induced body weight gain. FIG. 2A: Male C57BL/6J mice were fed a control or high-fat diet supplemented with or without nervonic acid and body weights were followed over 12 weeks. Pairwise comparisons between groups using a permutation test was performed (n=10-12 mice/group * p<0.05, *** p<0.001). FIG. 2B: Body composition was assessed from the same mice by NMR-MRI. One-way ANOVA was utilized to assess differences between groups (n=10-12 mice/group *** p<0.001 from control, §§§ p<0.001 from HFD, § p=0.025 from HFD). 2-way ANOVA demonstrated a significant interaction of diet with NA for fat mass (p<0.001). FIG. 2C: Average food consumption was measured over 12 weeks (n=10-12 mice/group *** p<0.001). FIG. 2D: Bomb calorimetry was utilized to quantify fecal caloric content (n=10-11 mice/group * p<0.05).

FIGS. 3A-3F: Nervonic acid-enrichment of a HFD leads to energy expenditure values similar to control mice. VO2 (FIG. 3A), VCO2 (FIG. 3B), RER

(FIG. 3C), Heat (FIG. 3D), and locomotion (FIGS. 3E and 3F) were assessed by placing mice on indicated diets in metabolic cages. Shaded region in FIG. 3E represents the dark cycle (n=10-12 mice/group ** p<0.01, *** p<0.001). 2-way ANOVA demonstrates a significant interaction of diet with NA for V02, VCO2, and heat in the light cycle (p<0.05).

FIGS. 4A-4F: Nervonic acid improves glucose tolerance and insulin sensitivity in mice on a high fat diet. Fasting (FIG. 4A) and random-fed (FIG. 4B) blood glucose, fasting insulin (FIG. 4C) and insulin (FIG. 4D) levels after IP injection of glucose at indicated time points were measured in mice after 8 weeks on different diets. GTT (FIG. 4E) and ITT (FIG. 4F) were performed on mice after 10 and 11 weeks on diets, respectively. One-way ANOVA was utilized to assess differences between groups (n=10-12 mice/group * p<0.05, ** p<0.01, *** p<0.001). 2-way ANOVA demonstrates a significant interaction of diet with NA for fasting blood glucose (p<0.05), fasting insulin (p<0.001), glucose-stimulated insulin production (p<0.05), GTT (p<0.01) and ITT (p<0.005).

FIGS. 5A-5D: Nervonic acid enrichment improves markers of liver fatty acid oxidation. FIG. 5A: qRT-PCR was utilized to assess transcript levels of PPARα, PGC1α, and SIRT1 (n=8 mice/group * p<0.05, ** p<0.01). Short-chain (FIG. 5B), medium-chain (FIG. 5C), and long-chain-chain (FIG. 5D) acylcarnitine levels were assessed by LC-MS/MS (n=10-11 mice/group * p<0.05, ** p<0.01, *** p<0.001 when compared to control, #p<0.05, ##p<0.01, ###p<0.001 for HFD to HFD+NA comparisons). Where significant, results from 2-way ANOVA indicating an interaction between the diet and NA are indicated above each acylcarnitine (● p<0.05, ●● p<0.01, ●●● p<0.001).

### DETAILED DESCRIPTION

Lipid perturbations contribute to detrimental outcomes in obesity. It has previously been demonstrated that nervonic acid, a C24:1 ω-9 fatty acid, predominantly acylated to sphingolipids, including ceramides, are selectively reduced in a mouse model of obesity. It is currently unknown if deficiency of nervonic acid-sphingolipid metabolites contribute to complications of obesity.

As disclosed herein, mice were fed a standard diet, a high fat diet, or these diets supplemented isocalorically with nervonic acid. An objective was to determine if dietary nervonic acid content alters the metabolic phenotype in mice fed a high fat diet.

Furthermore, if nervonic acid alters markers of impaired fatty acid oxidation in the liver was investigated. The presently disclosed subject matter provides that a nervonic acid-enriched isocaloric diet reduced weight gain and adiposity in mice fed a high fat diet. The nervonic acid enrichment led to increased C24:1-ceramides and improved several metabolic parameters including blood glucose levels, and insulin and glucose tolerance. Mechanistically, nervonic acid supplementation increased PPARα and PGC1α expression and improved the acylcarnitine profile in liver. These alterations indicated improved energy metabolism through increased β-oxidation of fatty acids. Taken together, increasing dietary nervonic acid improved metabolic parameters in mice fed a high fat diet. Strategies that prevent deficiency of and/or restore nervonic acid this represent an effective strategy to treat obesity and obesity-related complications.

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

A disease or disorder is “alleviated” if the severity of a symptom of the disease, condition, or disorder, or the frequency with which such a symptom is experienced by a subject, or both, are reduced.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

As used herein, an “analog” of a chemical compound is a compound that, by way of example, resembles another in structure but is not necessarily an isomer (e.g., 5-fluorouracil is an analog of thymine).

As used herein, “amino acids” are represented by the full name thereof, by the three letter code corresponding thereto, or by the one-letter code corresponding thereto, as indicated in Table 1:

TABLE 1
Amino Acids, Nomenclature, and Functionally Equivalent Codons

| | 3-Letter | 1-Letter | |
|---|---|---|---|
| Full Name | Code | Code | Functionally Equivalent Codons* |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

*Note:
codons are listed with respect to RNA nucleotides, but it would be understood that all instances of “U” in the listed codons would be “T” in the corresponding DNA sequence.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

The expression “amino acid” as used herein is meant to include both natural and synthetic amino acids, and both D and L amino acids. “Standard amino acid” means any of the twenty standard L-amino acids commonly found in naturally occurring peptides.

“Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

Synthetic or non-naturally occurring amino acids refer to amino acids which do not naturally occur in vivo but which, nevertheless, can be incorporated into the peptide structures described herein. The resulting “synthetic peptide” contain amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, α-amino acids such as L-α-hydroxylysyl and D-α-methylalanyl, L-α-methylalanyl, β-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as exchanges within one of the following five groups:

I. Small aliphatic, nonpolar or slightly polar residues: Ala, Ser, Thr, Pro, Gly;

II. Polar, negatively charged residues and their amides: Asp, Asn, Glu, Gln;

III. Polar, positively charged residues: His, Arg, Lys;

IV. Large, aliphatic, nonpolar residues: Met Leu, Ile, Val, Cys

V. Large, aromatic residues: Phe, Tyr, Trp

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, in some embodiments at least about 50%, and in some embodiments at least about 75%, in some embodiments at least about 90%, or in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting a risk or propensity to an addictive related disease disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

As used herein, an “essentially pure” preparation of a particular protein or peptide is a preparation wherein in some embodiments at least about 95% and in some embodiments at least about 99% by weight of the protein or peptide in the preparation is the particular protein or peptide.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993. This algorithm is incorporated into the NBLAST and XBLAST programs of Altschul et al., 1990, and can be accessed for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit”, as used herein when referring to a function, refers to the ability of a compound of the presently disclosed subject matter to reduce or impede a described function. In some embodiments, inhibition is by at least 10%, in some embodiments by at least 25%, in some embodiments by at least 50%, and in some embodiments, the function is inhibited by at least 75%. When the term “inhibit” is used more generally, such as “inhibit Factor I”, it refers to inhibiting expression, levels, and activity of Factor I.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting, or applying, or administering” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, or rectal approaches.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to, through ionic or hydrogen bonds or van der Waals interactions.

As used herein, the phrase “long chain fatty acid” refers to a fatty acid that has at least 22 carbons in its backbone. Exemplary long chain fatty acids include, but are not limited to C22, C24, and C26 fatty acids such as, but not limited to erucic acid, nervonic acid, and ximenic acid.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the phrase “omega 9 fatty acid” refers to an unsaturated fatty acid that are characterized by the final carbon-carbon double bond being in the omega 9 position (i.e., the ninth bond from the methyl end of the fatty acid). Exemplary omega 9 fatty acids include hypogeic acid (16:1 (n-9); also referred to as (Z)-hexadec-7-enoic acid), oleic acid (18:1 (n-9); also referred to as (Z)-octadec-9-enoic acid), elaidic acid (18:1 (n-9); also referred to as (E)-octadec-9-enoic acid), gondoic acid (20:1 (n-9); also referred to as (Z)-eicos-11-enoic acid), mead acid (20:3 (n-9); also referred to as (5Z,8Z,11Z)-eicosa-5,8,11-trienoic acid), erucic acid (22:1 (n-9); also referred to as (Z)-docos-13-enoic acid), nervonic acid (24:1 (n-9); also referred to as (Z)-tetracos-15-enoic acid), and ximenic acid (26:1 (n-9)).

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides but when used in the context of a longer amino acid sequence can also refer to a longer polypeptide.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of contracting the disease and/or developing a pathology associated with the disease.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., 1994).

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals. In some embodiments, a subject is a human.

As used herein, a “subject in need thereof” is a patient, animal, mammal, or human, who will benefit from the method of this presently disclosed subject matter.

As used herein, “substantially homologous amino acid sequences” includes those amino acid sequences that have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference amino acid sequence. Amino acid sequence similarity or identity can be computed by using the BLASTP and TBLASTN programs which employ the BLAST (basic local alignment search tool) algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Karlin & Altschul, 1993; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when it is in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine”, as used herein, is meant a composition which when inoculated into a subject has the effect of stimulating an immune response in the subject, which serves to fully or partially treat and/or protect the subject against a condition, disease or its symptoms. In one aspect, the condition is HIV. TB is another application as are parasitic diseases. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### II. Compositions

III.A. Long Chain Fatty Acids and Derivatives Thereof

In some embodiments, the presently disclosed subject matter relates to compositions comprising, consisting essentially of, or consisting of long chain fatty acid of at least 22 carbons and/or a derivative thereof. As used herein, the phrase “long chain fatty acid of 22 carbons” refers to any fatty acid of at least 22 carbons in its carbon backbone. Exemplary long chain fatty acids thus include erucic acid, nervonic acid, and ximenic acid. In some embodiments, a long chain fatty acid is an omega 9 fatty acid. In some embodiments, a long chain fatty acid is a monounsaturated omega 9 fatty acid.

Additionally, derivatives of long chain fatty acids can also be employed as disclosed herein. As used herein, the term “derivative” refers to a molecule that is based on a long chain fatty acid but has been modified or otherwise provided in a form that generates or is based on the long chain fatty acid. Exemplary long chain fatty acid derivatives include metabolic precursors, metabolites, analogs, esters, pharmaceutically acceptable salts, and any combination thereof.

In some embodiments, a derivative has undergone one or more chemical reactions to produce a new molecule. Such chemical reactions can include bioconjugations, which refer to the long chain fatty acid having been modified by attachment of one or more other biomoieties. Exemplary biomoieties that can be bioconjugated to long chain fatty acids for use in the compositions and methods of the presently disclosed subject matter include ceramides, lipids, including but not limited to phospholipids, cholesterols, glycerides, including but not limited to a diglycerides and triglycerides, monoacylglycerols, and glycerophopholipids. Exemplary bioconjugations can employ ester linkages, ether linkages, and amide linkages, among others.

In some embodiments, the long chain fatty acid is nervonic acid and the nervonic acid is selected from the group consisting of free nervonic acid, nervonic acid methyl ester, nervonic acid ethyl ester, and nervonic acid bioconjugated to one or more phospholipids, cholesterols, glycerides, including but not limited to a diglycerides and triglycerides, monoacylglycerols, and/or glycerophopholipids.

In some embodiments, the composition is administered as part of a nanoscale or microscale delivery vehicle, wherein the delivery vehicle is optionally selected from the group consisting of a liposome, a lipo/polymer, a microparticle, and a nanoparticle, or any combination thereof. In some embodiments, the delivery vehicle comprises a nanoliposome, and further wherein the nanoliposome encompasses the long chain fatty acid, the precursor thereof, the metabolite thereof, the analog thereof, the ester thereof, the pharmaceutically acceptable salt thereof, the combination thereof and/or comprises a lipid bilayer that comprises the long chain fatty acid, the precursor thereof, the metabolite thereof, the analog thereof, the ester thereof, the pharmaceutically acceptable salt thereof, the combination thereof. In some embodiments, the delivery vehicle is designed to degrade in the subject in order to release the long chain fatty acid of at least 22 carbons, the precursor thereof, the metabolite thereof, the analog thereof, the ester thereof, the pharmaceutically acceptable salt thereof, or any combination thereof to the subject over a period of time. In some embodiments, the delivery vehicle releases the long chain fatty acid of at least 22 carbons, the precursor thereof, the metabolite thereof, the analog thereof, the ester thereof, the pharmaceutically acceptable salt thereof, or any combination thereof to the subject's circulation and/or a cell, tissue, and/or organ of subject over the period of time. In some embodiments, the delivery vehicle is designed to degrade subsequent to contact with the subject's digestive system or circulatory system. In some embodiments, the delivery vehicle is designed to degrade in the subject to release at least about 50% of the long chain fatty acid of at least 22 carbons, the precursor thereof, the metabolite thereof, the analog thereof, the ester thereof, the pharmaceutically acceptable salt thereof, or any combination thereof over a period of time of at least 30 minutes, at least 1 hour, at least 6 hours, at least 12 hours, at least 24 hours, or longer than 24 hours.

In some embodiments, the presently disclosed subject matter provides for the use of compositions comprising liposomes. Liposomes can be prepared by any of a variety of techniques that are known in the art. See e.g., Betageri et al., 1993; Gregoriadis, 1993; Janoff, 1999; Lasic & Martin, 1995; and U.S. Pat. Nos. 4,235,871; 4,551,482; 6,197,333; and 6,132,766, each of which is incorporated herein by reference in its entirety. Temperature-sensitive liposomes can also be used, for example THERMOSOMES™ as disclosed in U.S. Pat. No. 6,200,598, which is incorporated herein by reference in its entirety. Entrapment of an active agent within liposomes of the presently disclosed subject matter can also be carried out using any conventional method in the art. In preparing liposome compositions, stabilizers such as antioxidants and other additives can be used.

Other lipid carriers can also be used in accordance with the presently disclosed subject matter, such as lipid microparticles, micelles, lipid suspensions, and lipid emulsions. See, e.g., Labat-Moleur et al., 1996; U.S. Pat. Nos. 5,011,634; 6,056,938; 6,217,886; 5,948,767; and 6,210,707, each of which is incorporated herein by reference in its entirety.

Delivery time frames can be provided according to a desired treatment approach. By way of example and not limitation, the first delivery vehicle can deliver substantially all of the provided active agent within 24 hours after administration wherein the second delivery vehicle can deliver a certain much smaller amount within the first 24 hours, first 3 days, first week, and substantially all within the first 2, 3, 4, 5, 6, or 7 weeks, as desired. Thus, the duration of the delivery can be altered with the chemistry of the delivery vehicle.

The delivery vehicles can comprise nano-, submicron-, and/or micron-sized particles. In some embodiments, the delivery vehicles are about 50 nm to about 1 m in their largest dimensions. Thus, in some embodiments the delivery vehicle can comprise a nanoparticle, a microparticle, or any combination thereof. As used herein, the terms “nano”, “nanoscopic”, “nanometer-sized”, “nanostructured”, “nanoscale”, and grammatical derivatives thereof are used synonymously and interchangeably and mean nanoparticles and nanoparticle composites less than or equal to about 1,000 nanometers (nm) in diameter. Similarly, the terms “micro”, “microscopic”, “micrometer-sized”, “microstructured”, “microscale”, and grammatical derivatives thereof are used synonymously and interchangeably and mean microparticles and microparticle composites that are larger than 1,000 nanometers (nm) but less than about 5, 10, 25, 50, 100, 250, 500, or 1000 micrometers in diameter.

The term “delivery vehicle” as used herein thus denotes a carrier structure which is biocompatible with and sufficiently resistant to chemical and/or physical destruction by the environment of use such that a sufficient amount of the delivery vehicles remain substantially intact after deployment at a site of interest. If the active agent is to enter a cell, tissue, or organ in a form whereby it is adsorbed to the delivery vehicle, the delivery vehicle must also remain sufficiently intact to enter the cell, tissue, or organ. Biodegradation of the delivery vehicle is permissible upon deployment at a site of interest.

As used herein, the term “biodegradable” means any structure, including but not limited to a nanoparticle, which decomposes or otherwise disintegrates after prolonged exposure to physiological conditions. To be biodegradable, the structure should be substantially disintegrated within a few weeks after introduction into the body.

Biodegradable biocompatible polymers can be used in drug delivery systems (Soppimath et al., 2001; Song et al., 1997; U.S. Patent Application Publication Nos. 2011/0104069, 2013/0330279, 2018/0078657, 2019/0091280, and 2020/0038452, and U.S. Pat. Nos. 7,332,586; 7,901,711; 8,137,697; 8,449,915; and 8,663,599, each of which is incorporated herein by reference in its entirety). The biodegradability and biocompatibility of poly(lactic acid) (PLA), poly(lactide-co-glycolide) (PLGA), and polyanhydrides (PAH) have been demonstrated. Some of the advantages of these materials include administration in high concentrations of the drug locally with low systemic levels, which reduces systemic complications and allergic reactions (Calhoun et al., 1997). Additionally, no follow-up surgical removal is required once the drug supply is depleted (Mandal et al., 2002). Biodegradation occurs by simple hydrolysis of the ester backbone in aqueous environments such as body fluids. The degradation products are then metabolized to carbon dioxide and water (de Faria et al., 2005). Several techniques have been developed to prepare nanoparticles loaded with a broad variety of drugs using PLGA and to some extent with PAH (Lamprecht et al., 1999; Astete et al., 2006; Hans et al., 2002; Kumar et al., 2004; Laurencin et al., 2001; Gonsalves et al., 1998; Kwon et al., 2001).

In some embodiments, the composition can comprise a pharmaceutically acceptable carrier, diluent, or excipient. As used herein, the term “pharmaceutically acceptable” and grammatical variations thereof, as it refers to compositions, carriers, diluents and reagents, means that the materials are capable of administration to or upon a vertebrate subject without the production of undesirable physiological effects such as nausea, dizziness, gastric upset, fever and the like. In some embodiments, the “pharmaceutically acceptable” refers to pharmaceutically acceptable for use in human beings.

Compositions in accordance with the presently disclosed subject matter generally comprise an amount of the desired delivery vehicle (which can be determined on a case-by-case basis), admixed with an acceptable pharmaceutical diluent or excipient, such as a sterile aqueous solution, to give an appropriate final desired concentration in accordance with the dosage information set forth herein, and/or as would be apparent to one of ordinary skill in the art upon a review of the instant disclosure, with respect to the antibiotic. Such formulations will typically include buffers such as phosphate buffered saline (PBS), or additional additives such as pharmaceutical excipients, stabilizing agents such as BSA or HSA, or salts such as sodium chloride. Such components can be chosen with the preparation of composition for local, and particularly topical, administration in mind.

III.B. Formulations

The compositions of the presently disclosed subject matter can be administered in any formulation or route that would be expected to deliver the compositions to the subjects and/or target sites present therein.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject. For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

III.C. Routes of Administration

By way of example and not limitation, suitable methods for administering a composition in accordance with the methods of the presently disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see e.g., U.S. Pat. No. 6,180,082, which is incorporated herein by reference in its entirety). In some embodiments, a composition comprising a nanoparticle and/or an exosome is administered orally.

Thus, exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated.

III.D. Dose

An effective dose of a composition of the presently disclosed subject matter is administered to a subject in need thereof. An “effective amount” or a “therapeutic amount” is an amount of a composition sufficient to produce a measurable response. Exemplary responses include biologically or clinically relevant responses in subjects such as but not limited to an increase in insulin sensitivity, a inhibition of or reduction in obesity, an improvement in a metabolic-related disorder or a symptom thereof, etc. Actual dosage levels of the compositions of the presently disclosed subject matter can be varied so as to administer an amount of the composition that is effective to achieve the desired response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore an “effective amount” can vary. However, using the methods described herein, one skilled in the art can readily assess the potency and efficacy of a composition of the presently disclosed subject matter and adjust the regimen accordingly.

As such, after review of the instant disclosure, one of ordinary skill in the art can tailor the dosages to an individual subject, taking into account the particular formulation, method of administration to be used with the composition, and particular disease, disorder, and/or condition treated or biologically relevant outcome desired. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art.

### III. Methods and Uses

III.A. Methods for Preventing and/or Reducing Weight Gain and/or for Preventing and/or Reducing Development of Obesity

In some embodiments, the presently disclosed subject matter provides methods for preventing and/or reducing weight gain in subjects, optionally mammals, further optionally humans. In some embodiments, the methods comprise, consist essentially of, or consist of administering to the subject, mammal, or human an effective amount of a composition as disclosed herein, wherein the effective amount of the composition is effective for preventing or reducing weight gain in the subject, mammal, or human relative to that which would have occurred in the subject, mammal, or human in the absence of the composition.

In some embodiments, the presently disclosed subject matter also provides methods for preventing and/or reducing the development of obesity in a subject, optionally a mammal, further optionally a human. In some embodiments, the methods comprise, consist essentially of, or consist of administering to the subject an effective amount of a composition as disclosed herein, wherein the effective amount of the composition prevents and/or reduces the development of obesity in the subject.

Thus, in some embodiments of the presently disclosed methods, the composition to be administered comprises, consists essentially of, or consists of one or more long chain fatty acid of at least 22 carbons and/or derivatives thereof. In some embodiments, the derivatives are metabolic precursor thereof, metabolites thereof, analogs thereof, esters thereof, ethers thereof, amides thereof, pharmaceutically acceptable salts thereof, or any combination thereof. In some embodiments, the long chain fatty acid is an omega 9 fatty acid, which in some embodiments can be a monounsaturated omega 9 fatty acid. In some embodiments, the long chain fatty acid is selected from the group consisting of erucic acid, nervonic acid, and ximenic acid.

In some embodiments, the derivative of the of long chain fatty acid of at least 22 carbons is a metabolic precursor thereof, a metabolite thereof, an analog thereof, an ester thereof, an ether thereof, an amide thereof, a pharmaceutically acceptable salt thereof, or any combination thereof. In some embodiments, the derivative comprises the long chain fatty acid bioconjugated to a biomolecule selected from the group consisting of a ceramide, a lipid, a phospholipid, a cholesteroal, a dyglyceride, a triglyceride, a monoacylglycerol, and a glycerophopholipid. In some embodiments, the long chain fatty acid if bioconjugated to the biomolecule via an ester linkage, an ether linkage, an amide linkage, or any combination thereof. In some embodiments, the derivative of the long chain fatty acid is a methyl ester, an ethyl ester, or any combination thereof. In some embodiments, the composition administered comprises, consists essentially of, or consists of nervonic acid, optionally an ester thereof, and further optionally a methyl ester and/or an ethyl ester thereof. In some embodiments, the composition comprises, consists essentially of, or consists of nervonic acid and/or a derivative thereof bioconjugated to a ceramide, optionally wherein the derivative thereof is a nervonic acid ethyl ester.

III.B. Methods for Preventing and/or Reducing Weight Gain and/or for Inhibiting Reduction of Very-Long Chain Lipids in Subjects

As disclosed herein, the presently disclosed subject matter also provides methods for inhibiting reduction of very-long chain lipids such as but not limited to sphingolipids in subjects. In some embodiments, the methods comprise, consist essentially of, or consist of administering to a subject in need thereof an effective amount of a composition as disclosed herein, wherein the effective amount of the composition is effective to inhibit reduction of very-long chain sphingolipids in the subject. In some embodiments, the composition administered comprises, consists essentially of, or consisting of long chain fatty acid of at least 22 carbons, a metabolic precursor thereof, a metabolite thereof, an analog thereof, an ester thereof, an ether thereof, an amide thereof, a pharmaceutically acceptable salt thereof, or any combination thereof. In some embodiments, the long chain fatty acid is a monounsaturated omega 9 fatty acid. In some embodiments, the long chain fatty acid is selected from the group consisting of erucic acid, nervonic acid, and ximenic acid. In some embodiments, the long chain fatty acid is C24:1 nervonic acid. In some embodiments, the ester thereof is a C₁-C₆ straight chain ester, C₁-C₆ branched chain ester, or any combination thereof. In some embodiments, the ester thereof is a methyl ester, an ethyl ester, or any combination thereof.

In some embodiments of the presently disclosed methods, the composition comprises, consists essentially of, or consists of nervonic acid, optionally an ester thereof, and further optionally a methyl ester and/or an ethyl ester thereof. In some embodiments, the composition comprises, consists essentially of, or consists of a nervonic acid ethyl ester bioconjugated to a ceramide.

As disclosed herein, consuming a high fat diet can lead to a reduction in very-long chain sphingolipids. As such, in some embodiments, the reduction in very-long chain sphingolipids is a consequence of the mammal consuming a high fat diet. In some embodiments, the subject is a human.

III.C. Methods for Modulating Lipid and/or Ceramide Contents in Subjects

In some embodiments, the presently disclosed subject matter also provides methods for increasing content of one or more first species of lipids in a subject while simultaneously decreasing content of one or more second species of C20-C26 ceramides in the subject by administering an effective amount of one or more compositions as disclosed herein, wherein the effective amount is effective for increasing content of the one or more first species of ceramides in the mammal while simultaneously decreasing content of the one or more second species of C20-C26 ceramides in the mammal. In some embodiments, the one or more first species of lipids increased are selected from the group consisting of C24:1-lysophosphatidylcholine an diacylglycerides C16:0/C24:1, C18:0/C24:1, C18:1/C24:1, and C18:2/C24:1. In some embodiments, wherein the content is plasma lipid and/or ceramide content, liver lipid and/or ceramide content, or both.

III.D. Methods for Reducing Blood Glucose Levels Resulting from Consumption of High Fat Diets in Subjects

The presently disclosed subject matter also relates in some embodiments to methods for reducing blood glucose levels resulting from consumption of a high fat diet in subjects in need thereof, wherein the methods comprise, consist essentially of, or consist of administering to the subject an effective amount of a composition as disclosed herein, wherein the effective amount is effective for reducing the blood glucose level in the subject. In some embodiments, the composition thus comprises, consists essentially of, or consists of long chain fatty acid of at least 22 carbons or a derivative thereof such as but not limited to a metabolic precursor thereof, a metabolite thereof, an analog thereof, an ester thereof, an ether thereof, an amide thereof, a pharmaceutically acceptable salt thereof, or any combination thereof. In some embodiments, the long chain fatty acid is a monounsaturated omega 9 fatty acid and/or is selected from the group consisting of erucic acid, nervonic acid, and ximenic acid.

In some embodiments, the composition comprises, consists essentially of, or consists of nervonic acid, optionally an ester thereof, and further optionally a methyl ester and/or an ethyl ester thereof. In some embodiments, the long chain fatty acid is nervonic acid and the nervonic acid is selected from the group consisting of free nervonic acid, nervonic acid methyl ester, nervonic acid ethyl ester, and nervonic acid bioconjugated to a biomolecule selected from the group consisting of a ceramide, a lipid, a phospholipid, a cholesteroal, a dyglyceride, a triglyceride, a monoacylglycerol, and a glycerophopholipid.

In some embodiments of any of the presently disclosed methods, the composition can be administered as part of a nanoscale or microscale delivery vehicle, wherein the delivery vehicle is optionally selected from the group consisting of a liposome, a lipo/polymer, a microparticle, and a nanoparticle, or any combination thereof. In some embodiments, the delivery vehicle comprises a nanoliposome, and further wherein the nanoliposome encompasses the long chain fatty acid, the precursor thereof, the metabolite thereof, the analog thereof, the ester thereof, the pharmaceutically acceptable salt thereof, the combination thereof and/or comprises a lipid bilayer that comprises the long chain fatty acid, the precursor thereof, the metabolite thereof, the analog thereof, the ester thereof, the pharmaceutically acceptable salt thereof, the combination thereof. In some embodiments, the delivery vehicle is designed to degrade in the subject in order to release the long chain fatty acid of at least 22 carbons, the precursor thereof, the metabolite thereof, the analog thereof, the ester thereof, the pharmaceutically acceptable salt thereof, or any combination thereof to the subject over a period of time. In some embodiments, the delivery vehicle releases the long chain fatty acid of at least 22 carbons, the metabolic precursor thereof, the metabolite thereof, the analog thereof, the ester thereof, the ether thereof, the amide thereof, the pharmaceutically acceptable salt thereof, or any combination thereof to the subject's circulation and/or a cell, tissue, and/or organ of subject over the period of time. In some embodiments, the delivery vehicle is designed to degrade subsequent to contact with the subject's digestive system or circulatory system.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the EXAMPLES

Animals. Animal experiments were approved by the Animal Care and Use Committee of the University of Virginia (Charlottesville, Va., United States of America). C57Bl/6J mice were obtained from Jackson Laboratories (Bar Harbor, Me., United States of America). Mice were group housed in standard cages with up to five mice per cage with a 12 hour dark/light cycle. All mice were weighed once per week. Weekly food consumption was measured, with the amount consumed per cage averaged amongst the mice in the cage. Body composition was determined by use of an ECHOMRI™-100H Body Composition Analyzer (EchoMRI LLC, Houston, Tex., United States of America).

Diets. Diets were obtained from Research Diets (New Brunswick, N.J., United States of America). The low fat chow was D12450Ji (10 kcal % fat) and the high fat chow was D12492 (60 kcal % fat). These two chows were reformulated by replacing a portion of dietary fat with an ethyl ester of nervonic acid from Nu-Chek Prep (Elysian, Minn., United States of America) to produce isocaloric diets with nervonic acid at 6 g per kg of diet (0.6%). For the three most prevalent fatty acids in these diets: C16 comprised 14.4%, 11.7%, 18.5%, and 18.1%; C18:1 comprised 27.4%, 22.8%, 32%, and 31.4%; and C18:2 comprised 39.6%, 36.2%, 26.9%, and 26.5% of the fatty acid composition of the Cnt, Cnt+NA, HFD, and HFD+NA diets, respectively. The full fatty acid profile is shown in Table 1. Mice were started on diets at ˜8 weeks of age and given ad libitum access.

TABLE 1
Fatty Acid Profiles of Various Diets Employed

| | LFD (%) | LFD + NA (%) | HFD (%) | HFD + NA (%) |
|---|---|---|---|---|
| C10 | 0.044444 | 0.030444 | 0.090741 | 0.089019 |
| C12 | 0.044444 | 0.030444 | 0.090741 | 0.089019 |
| C14 | 0.544444 | 0.390444 | 1.007407 | 0.988463 |
| C15 | 0.044444 | 0.030444 | 0.090741 | 0.089019 |
| C16 | 14.35556 | 11.65356 | 18.47593 | 18.14354 |
| C16:1 | 0.677778 | 0.481778 | 1.27963 | 1.255519 |
| C17 | 0.233333 | 0.177333 | 0.372222 | 0.365333 |
| C18 | 6.877778 | 5.393778 | 9.97963 | 9.797074 |
| C18:1 | 27.4 | 22.794 | 31.98333 | 31.41672 |
| C18:2 | 39.62222 | 36.20622 | 26.93704 | 26.51681 |
| C18:3 | 4.688889 | 4.506889 | 1.864815 | 1.842426 |
| C20 | 0.311111 | 0.283111 | 0.218519 | 0.215074 |
| C20:1 | 0.433333 | 0.349333 | 0.572222 | 0.561889 |
| C20:2 | 0.355556 | 0.243556 | 0.725926 | 0.712148 |
| C20:3 | 0.044444 | 0.030444 | 0.090741 | 0.089019 |
| C20:4 | 0.133333 | 0.091333 | 0.272222 | 0.267056 |
| C22 | 0.166667 | 0.166667 | 0.027778 | 0.027778 |
| C22:5 | 0.044444 | 0.030444 | 0.090741 | 0.089019 |
| C24 | 0.111111 | 0.111111 | 0.018519 | 0.018519 |
| C24:1 | 0.0 | 14.0 | 0.0 | 1.722222 |

Insulin Tolerance Test (ITT) and Glucose Tolerance Test (GTT) and analysis of blood glucose and plasma insulin. ITT and GTT were performed as described in Lansey et al., 2012. Briefly, for ITT, random-fed mice were given an intraperitoneal injection (IP) of insulin (0.75 U/kg in 0.9% NaCl). Blood glucose levels were determined by a glucometer (Contour Next EZ Blood Glucose Monitoring System, Bayer, Leverkusen, Germany) at the indicated time points after injection. For GTT, mice were fasted for 16 hours prior to testing. D-Glucose, prepared the day before, was administered via an IP injection (1 g/kg), and blood glucose levels were determined by a glucometer at the indicated time points after injection. Plasma insulin levels were determined using a STELLUX® brand Chemi Rodent Insulin ELISA kit (Cat. No. 80-INSMR-CHO1; APLCO, Salem, N.H., United States of America) or an Ultrasensitive Rat/Mouse Insulin ELISA kit, Low Range Assay (Cat. No. 90060; Crystal Chem, Downers Grove, Ill., United States of America). The luminescence assay was conducted using a VICTOR2 Plate Reader (Perkin Elmer, Waltham, Mass., United States of America).

For assessment of insulin, blood was collected with heparinized capillary tubes and subsequently centrifuged to obtain plasma. Insulin levels was then determined using a STELLUX® Chemi Rodent Insulin ELISA kit (Catalog No. 80-INSMR-CHO1, APLCO, Salem, N.H., United States of America) or an Ultrasensitive Rat/Mouse Insulin ELISA kit low range (Cat. No 90060; Crystal Chem, Downers Grove, Ill., United States of America). The luminescence assay was conducted using a VICTOR2 Plate Reader (Perkin Elmer, Waltham, Mass., United States of America).

### Determination of Energy Expenditure, Respiratory Exchange Ratio, and Locomotion

For determination of energy expenditure, respiratory exchange ratios, and activity, mice were placed in an Oxymax metabolic chamber systems (Comprehensive Laboratory Animal Monitoring System, CLAMS, from Columbus Instruments (Columbus, Ohio, United States of America). Oxygen consumption (V02) and carbon dioxide production (VCO2) were determined for each mouse every 18 minutes over 72 hours with ambulatory activity assessed every minute over this same time period. Only the last two complete light and dark cycles (spanning 48 hours) were used for data analysis. Feces was collected from these studies and shipped to the Mouse Metabolic Phenotyping Core at the University of Texas Southwestern for bomb calorimetry analysis using a Parr 6200 Isoperibol Calorimeter (Parr Instrument Company, Moline, Ill., United States of America).

Cell culture. HEK293 cells were grown in DMEM containing 10% FBS. Cells were transfected with Silencer Select siRNA to CerS2 (s26789) or Silencer Select negative control No. 1 using LIPOFECTAMINE® 3000 brand transfection reagent following the manufacturer's protocol. All culture reagents were obtained from Thermo Fisher Scientific (Waltham, Mass., United States of America). 48 hours post-transfection, cells were treated with vehicle or 1 μM nervonic acid for 24 hours.

Transcript analysis. RNA was isolated from mouse liver by manual homogenization in TRIzol reagent (Invitrogen, Carlsbad, Calif., United States of America) following the manufacturer's instructions. Total RNA underwent DNase I treatment (New England Biolabs, Ipswich, Mass., United States of America) and cDNA was made using the ISCRIPT™ brand cDNA Synthesis Kit (Bio-Rad Laboratories, Hercules, Calif., United States of America). Quantitative real-time PCR was carried out using Bio-Rad SYBR® green probes (PPARα Bio-Rad Assay ID qMmuCID0005156; SIRT1 Bio-Rad Assay ID qMmuCID0015511; PGC1α Bio-Rad Assay ID qMmuCID0006032). The TATA binding protein (Bio-Rad Assay ID qMmuCID0040542) gene was used to normalize target gene abundance. Biological samples were run in triplicate.

Western Blotting. Liver protein lysates were isolated using RIPA lysis buffer as previously described. Protein quantification was completed using the DC protein assay (Bio-Rad). Protein samples were prepared by heating at 70° C. for 10 minutes after the addition of denaturing sample buffer. Proteins were separated using SDS-PAGE on a 4-12% gel (Thermo Fisher) and transferred to a nitrocellulose membrane. Antibodies were diluted in 5% BSA in Tris-buffered saline/Tween-20. After 1 hour of blocking in 5% BSA, membranes were incubated with the primary antibody, washed, incubated with a detectably-labeled secondary antibody, and then washed again. Protein bands were visualized. The following antibodies were used: anti-caveolin-1 (Catalog No. sc-7875; Santa Cruz Biotechnology, Santa Cruz, Calif., United States of America), anti-EGF receptor (Catalog No. 4267; Cell Signaling Technology, Inc. Danvers, Mass., United States of America), and anti-3-actin (Catalog No. A-5441; Sigma-Aldrich Corp., St. Louis, Mo., United States of America).

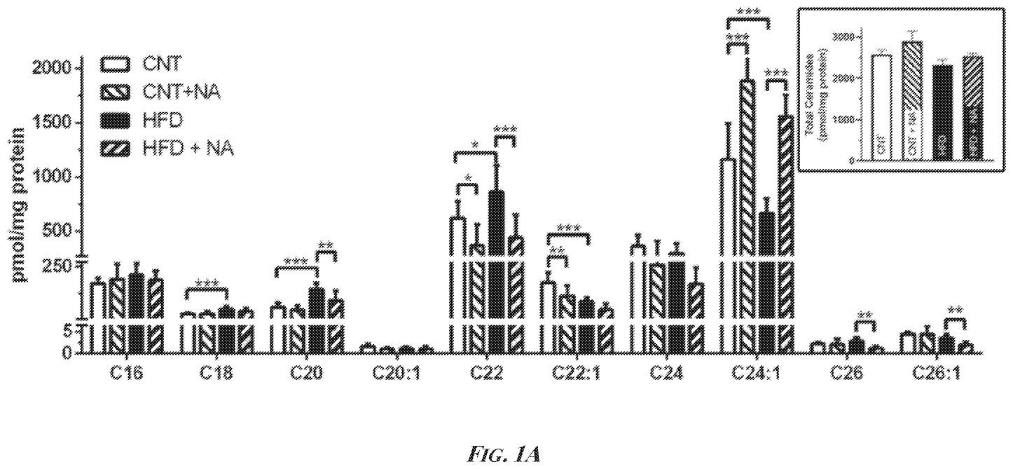

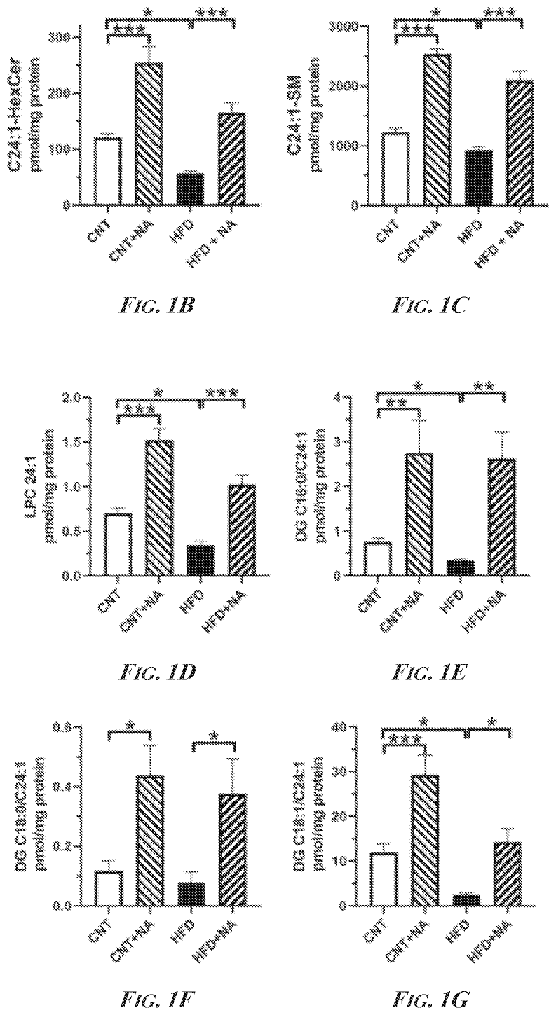

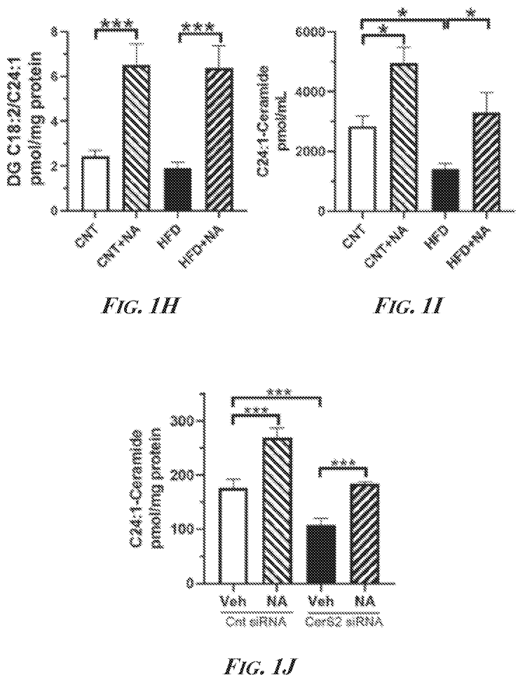

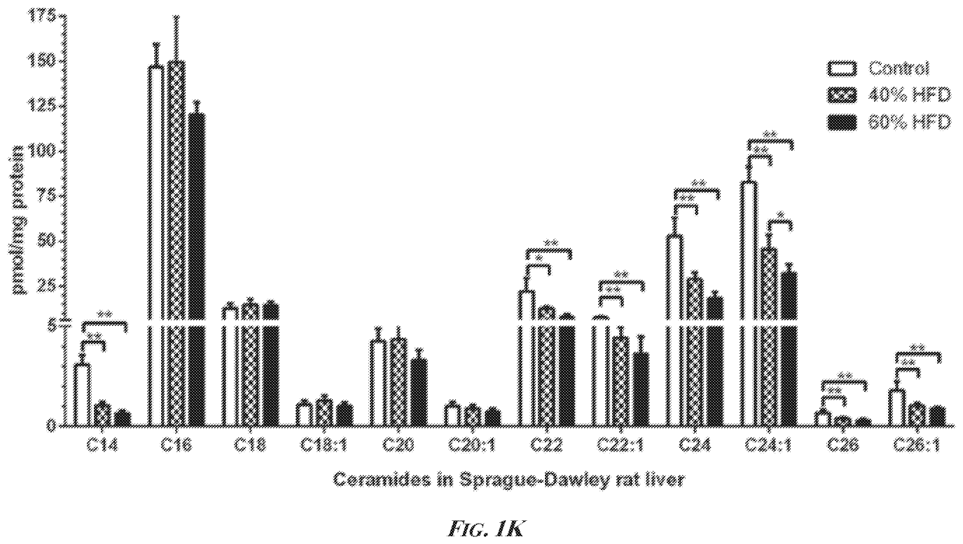

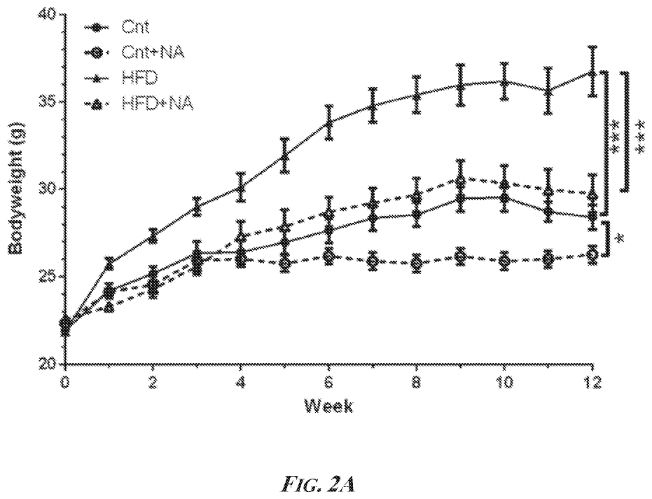

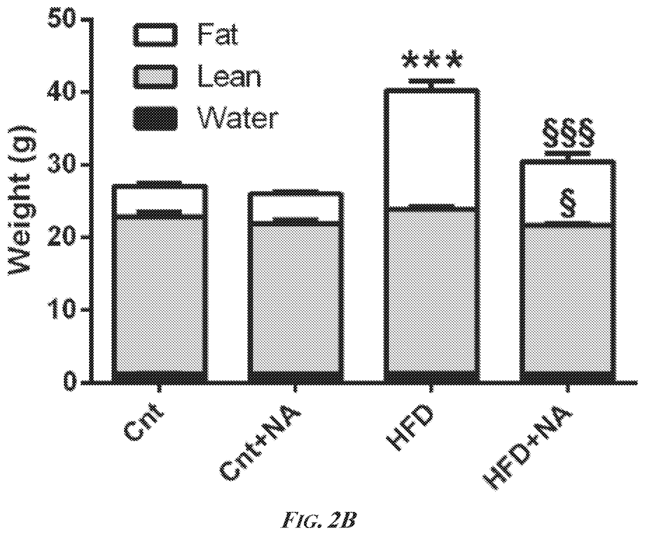

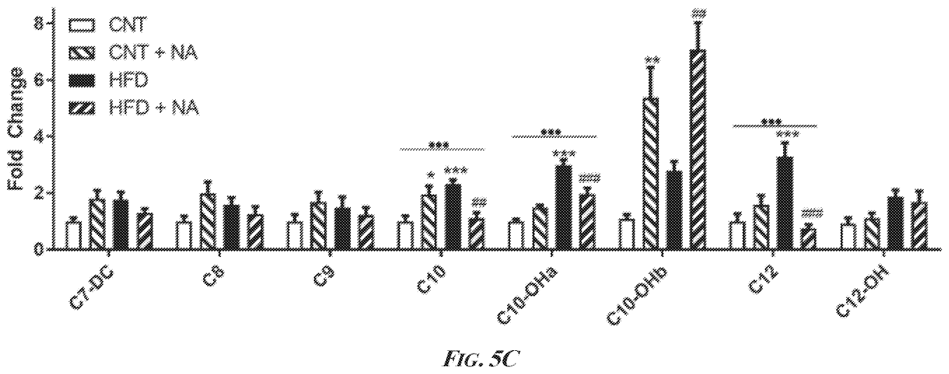

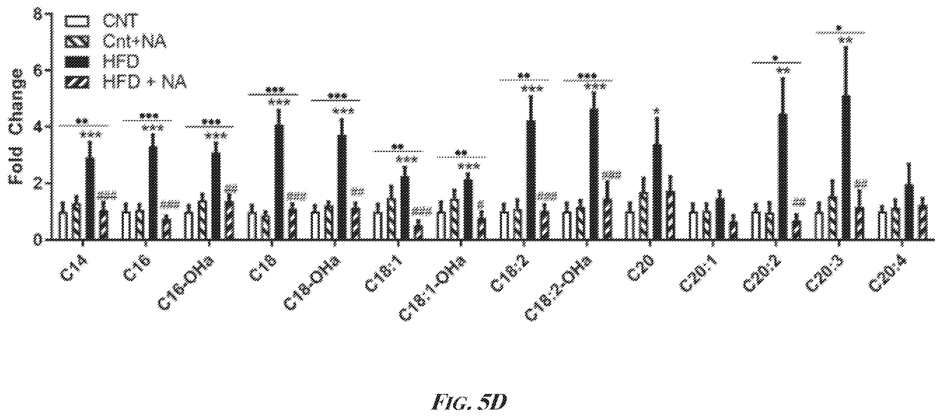

Sphingolipid and acylcarnitine measurements by liquid chromatography-mass spectrometry. Sphingolipids were analyzed as described previously (Wijesinghe et al., 2010) for FIG. 1B or with modifications described herein. Liver samples from rats fed a low or high fat diet were obtained from Scot Kimball's laboratory at the Pennsylvania State University (Hershey, Pa.). Mouse liver samples were analyzed with modifications as described previously (Pearson et al., 2020). For FIGS. 1A and 5D, liver homogenates were subjected to lipid extraction and subjected to liquid chromatography-electrospray ionization mass spectrometry was performed on an I-class Acquity with a 2.1 mm×10 cm C18 CSH 1.7 m particle size column coupled to an in-line TQ-S mass spectrometer from Waters Corporation (Milford, Mass., United States of America).

Acylcarnitines were quantified as their butyl esters as described in Giesbertz et al., 2015. Briefly, liver tissue was extracted in ice-cold methanol spiked with stable-isotopically labeled internal standards (Cambridge Isotope Laboratories, Tewksbury, Mass., United States of America). Insoluble material was pelleted and the supernatant subsequently dried down under vacuum. Samples were solubilized in n-butanol containing 5% acetyl chloride and incubated for 60° C. for 20 minutes, Samples were subsequently dried down again and resuspended in 50:50 water:methanol for LC-ESI-MS/MS analysis. LC-MS/MS analysis was performed on a I-class Acquity with a 2.1 mm×10 cm C18 CSH 1.7 m particle size column coupled to an in-line TQ-S mass spectrometer using multiple reaction monitoring. Calibration curves were generated from standard mixtures of acylcarnitines from Cambridge Isotope Laboratories.

Data and statistical analysis. Statistical analysis was performed with R or Graph-Pad Prism version 6 or 7 software (GraphPad Software, San Diego, Calif., United States of America). Statistical significance was determined by one-way ANOVA with Tukey's or Sidak's post-test for multiple comparison. Two-way ANOVA was utilized to test for interactions between diets and NA. Growth curves were analyzed using permutation tests of differences between groups of growth curves with the growth curve function of statmod in R. The number of permutations was set to 10,000. Thresholds of significance are indicated in Figure legends and represent adjusted p values. All data are expressed as mean±SEM with the number of replicates given in the Figure legends.

### Example 1

### High Fat Diet Reduced Very-long Chain Sphingolipids in a Rodent Model of Obesity

The ceramide profiles of livers of mice on a high fat diet (HFD) with 60% of calories from fat (soybean oil supplemented with lard) or a low fat chow (10 kcal % fat; see FIG. 1A) were examined. Two additional mouse groups were fed either an isocaloric normal or a HFD both enriched in an ethyl ester form of nervonic acid (NA, 0.6% by diet weight). In both the normal and HFD fed groups, NA-enrichment led to increases in C24:1-ceramides, with concomitant decreases in other ceramides C20-C26. The sum of the amounts of these individual ceramide species (FIG. 1A inset) demonstrated significant differences between diets. The HFD also reduced levels of C24:1-hexosylceramide (FIG. 1B) and C24:1-sphingomyelin (FIG. 1C) with dietary NA increasing these on both diets. As expected, only small amounts of NA-containing lipids were detected within glycerolipids, but a similar pattern was observed. The HFD reduced C24:1-lysophosphatidylcholine (FIG. 1D), diacylglycerides C16:0/C24:1 (FIG. 1E) and C18:1/C24:1 (FIG. 1G), and NA-enriched control and HFDs elevated these species, including diacylglycerides C18:0/C24:1 (FIG. 1F) and C18:2/C24:1 (FIG. 1H). C24:1-acylation was undetectable in other lipid classes (e.g. phosphatidic acid, phosphatidylglycerol). The HFD reduced C24:1-ceramides in plasma (FIG. 1I). Dietary NA-enrichment led to significant increases in plasma C24:1-ceramides on both the control and HFDs. Furthermore, siRNA-mediated knockdown of CerS2 in HEK293 cells demonstrated a similar pattern as observed in the liver. Cers2 knockdown diminished C24:1-ceramide amounts and reduced NA acylation into ceramide (FIG. 1J).

To assess the specificity of the findings in mice, we also determined the ceramide composition in livers of rats fed a 40% or 60% HFD (FIG. 1K). Decreases in steady-state C14, C22, C22:1, C24, C24:1, C26, and C26:1-ceramides in both 40% and 60% HFD fed rats was observed. More specifically, an approximate 55% reduction in C24:1- and C24-ceramides on the 40% HFD and an approximate 65% reduction on the 60% HFD was observed. Taken together, in two distinct rodent models of obesity, perturbations in very-long chain fatty acid-containing ceramides was observed, with diminished C24:1-ceramide being consistent in both models. These findings also demonstrated that a NA-enriched diet modulated ceramide profiles and increased C24:1-lipids in mice on either diet.

### Example 2

### Dietary Enrichment with Nervonic Acid Reduced Weight Gain in Mice Fed a High Fat Diet

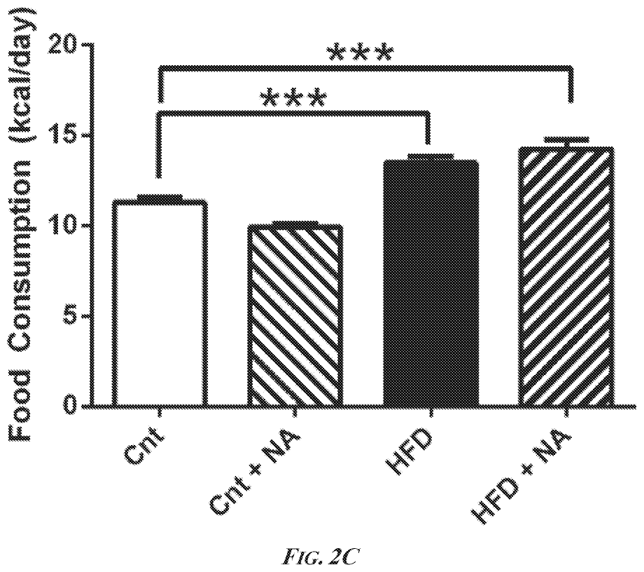

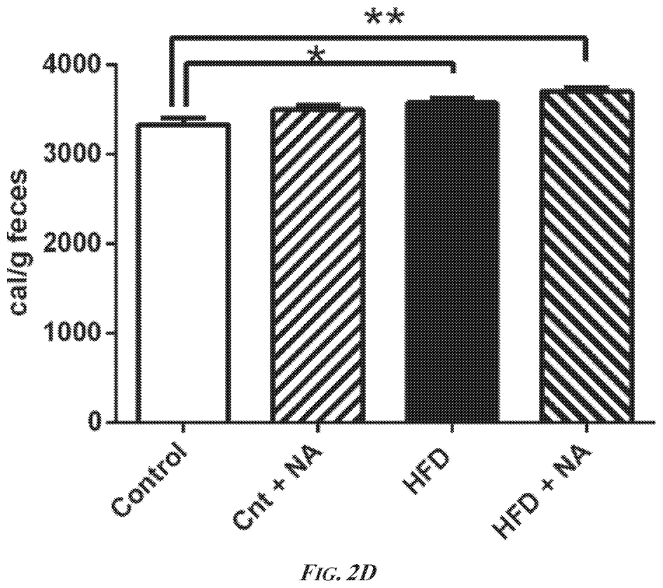

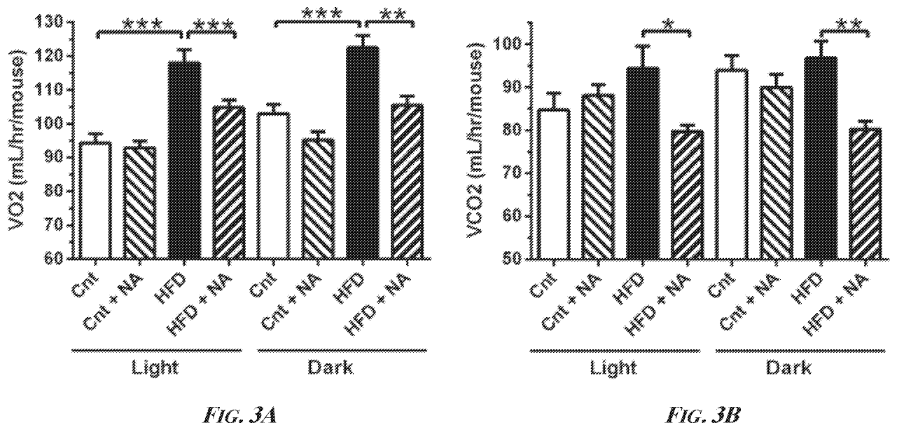

Mice were either fed normal, a 60% high fat, or NA-supplemented isocaloric normal or high fat chows and body weights, and were monitored over a three month period (FIG. 2A). As expected, the HFD fed mice gained more body weight than control diet fed mice. Interestingly, mice fed a HFD enriched with NA (HFD+NA) demonstrated significantly less body weight gain than the HFD fed mice, and body weights were not significantly different from mice on the control diet. Even mice fed a NA-enriched control diet demonstrated significantly less body weight gains over time than mice on the control diet.

Body composition analysis revealed that the difference in weight gain between mice on the HFD and HFD+NA could be attributed to a large extent to a reduction in fat mass in HFD+NA fed mice (FIG. 2B). At the time of sacrifice, HFD and HFD+NA fed animals showed average body fat contents of 16.44 g/mouse and 9.48 g/mouse, respectively. Lean mass was slightly, but significantly, reduced in the HFD+NA versus the HFD group, though the lean mass of the HFD+NA group was not significantly different to the lean mass of control or control+NA groups. The lean mass was also not significantly different between the control and control+NA diet groups. Water mass was similar for all experimental groups.

Throughout the duration of the study, average food consumption was determined (FIG. 2C). Mice on high fat diets demonstrated significant increases in kcal per day consumed compared to controls, while the average food consumption between HFD and HFD+NA was similar over the 12 weeks of the study. Food consumption of mice on the control+NA diet was also not significantly different from control mice.

To evaluate whether food calories were not equally well absorbed, fecal caloric measurements were determined by bomb calorimetry (FIG. 2D). Comparing gross heat of combustion, small but significant increases in fecal caloric content was observed in mice on HFD and HFD+NA diets compared to controls. No differences in fecal caloric content were observed between control and control+NA or HFD and HFD+NA diets.

Therefore, similar to food consumption, dietary NA enrichment did not have an apparent effect on diet absorption. Taken together, these data demonstrated that animals on a HFD supplemented with NA exhibit reduced body weight gain with reduced adiposity that was not due to reduced food consumption or absorption.

### Example 3

### Dietary Nervonic Acid Enrichment Improved Glycemic Control in Mice Fed a High Fat Diet

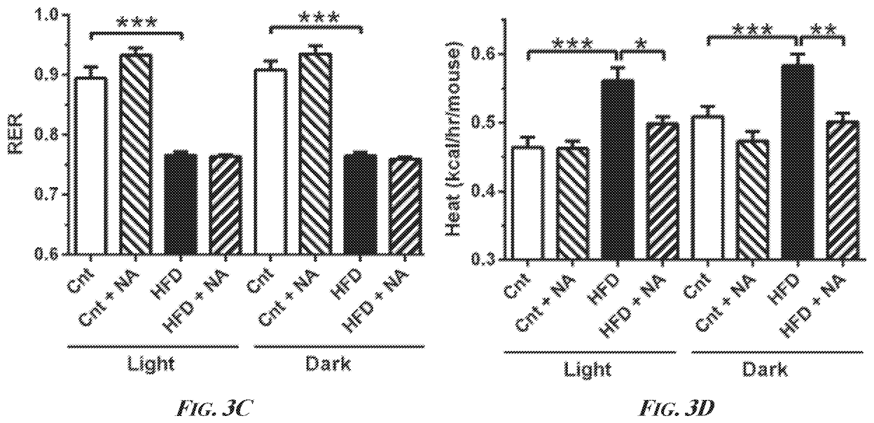

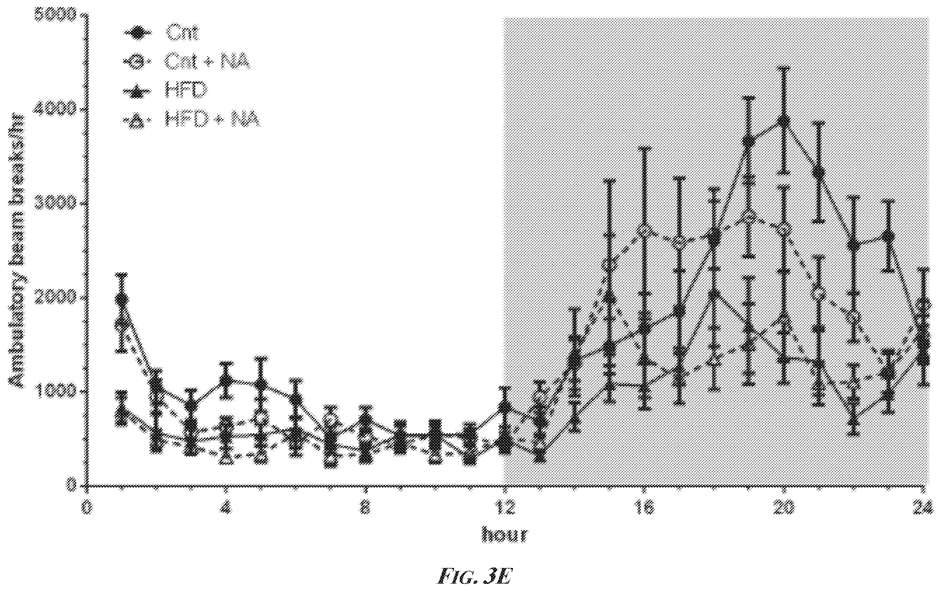

To determine the physiological consequence of nervonic acid enrichment, several parameters of glucose and insulin were assayed. Random-fed (FIG. 3A) and fasting (FIG. 3B) blood glucose measurements were determined. After 8 weeks, the high fat diet led to increased random-fed and fasting blood glucose levels compared with control. Mice on a high fat fed diet with nervonic acid demonstrated a significant reduction of blood glucose levels compared to their high fat fed counterparts in both random-fed and fasted conditions. The blood glucose levels of mice on this HFD+NA diet were not significantly different than mice on the control diet under random fed or faster conditions. No significant differences were observed between mice on a control and mice on a control with NA diet.

Plasma insulin levels were measured after 8 weeks on the diets (FIG. 3C). On the control diet, nervonic acid did not influence insulin levels. Mice on a high fat diet demonstrated a significant increase in basal insulin levels, whereas mice on a high fat diet with nervonic acid demonstrated reduced insulin levels compared to the HFD that was not significantly different to the control diet. Insulin levels were also assessed after the intraperitoneal administration of glucose (FIG. 3D). Mice on the HFD showed a significant increase in insulin after 30 min, which was not observed in mice on HFD enriched with nervonic acid. No discernable differences in insulin levels were observed at the time points analyzed in the control groups.

Glucose tolerance tests (GTT) were performed after 10 weeks on the diets (FIG. 3E). An elevated glucose load above basal as determined by calculating the area under the curve, was observed in mice on the high fat diet compared to control diets. Mice on a high fat diet enriched with nervonic acid showed a reduction in the glucose load.

Insulin tolerance tests (ITT) were performed to assess insulin action (FIG. 3F). Mice on a high fat diet showed a diminished response to lower blood glucose levels after insulin injection, whereas mice on a high fat containing nervonic acid diet responded similarly to mice on the control diet. Mice on a control diet containing nervonic acid responded similar to the control diet.

### Example 4

### Dietary Nervonic Acid Altered Energy Expenditure Parameters but not Activity

As dietary consumption (FIG. 2C) or assimilation (FIG. 2D) did not appear to be contributing to the changes in weight gain, whether body weight changes of mice on nervonic acid enriched high fat diet compared to the high fat diet were a consequence of changes in energy homeostasis. The results are presented in FIG. 3.

Using metabolic cages, elevated oxygen consumption (V02; see FIG. 3A) was observed in mice on the HFD compared to controls in both the light and dark cycle. Mice on the HFD+NA had V02 levels that were significantly lower than in mice on a HFD and similar to mice on control diets in both the light and dark cycle. Carbon dioxide production (VCO2) in mice on the HFD was not significantly different from mice on control diets, but mice on the HFD+NA showed a significant reduction to control and HFD groups (FIG. 3B). When calculating respiratory exchange ratios (RER=VCO2/VO2) for both the light and dark cycle (FIG. 3C), it was determined that mice on the HFD and HFD+NA showed similar RERs and that these were significantly lower than for mice on control diets. These data indicated increased fat utilization for energy production in mice on high fat diets as expected. No statistical changes of RER was observed for mice on control+NA compared to the control diet for the light and dark, cycles. Heat production was highest in mice on HFD (FIG. 3D), while mice in the HFD+NA and control diet groups had similar lower values in both the light and dark cycles. No differences were observed between control and control+NA diets. Activity measurements were determined and graphed as beam breaks per hour (FIG. 3E) and cumulatively beam breaks over the 12-hour light and 12-hour dark cycles (FIG. 3F). Mice on a HFD demonstrated reduced ambulatory activity compared to mice on control diets. Ambulatory activities were similar for mice on the HFD and HFD+NA diets, and for mice on the control and control+NA diets.

Taken together, these data suggested that animals on a HFD, enriched with NA, have decreased energy expenditure and heat production, but exhibited similar ambulatory activity when compared to mice on the HFD. These data thus cannot explain the reduced body weight and adiposity in mice on the HFD+NA diet.

### Example 5

### Dietary Nervonic Acid Enrichment Improves Glycemic Control in Mice on a HFD

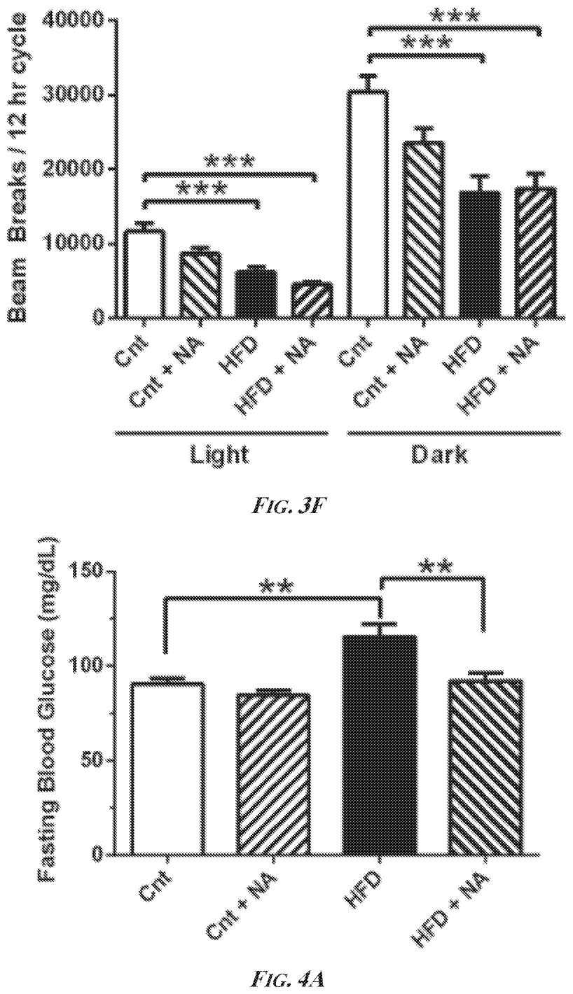

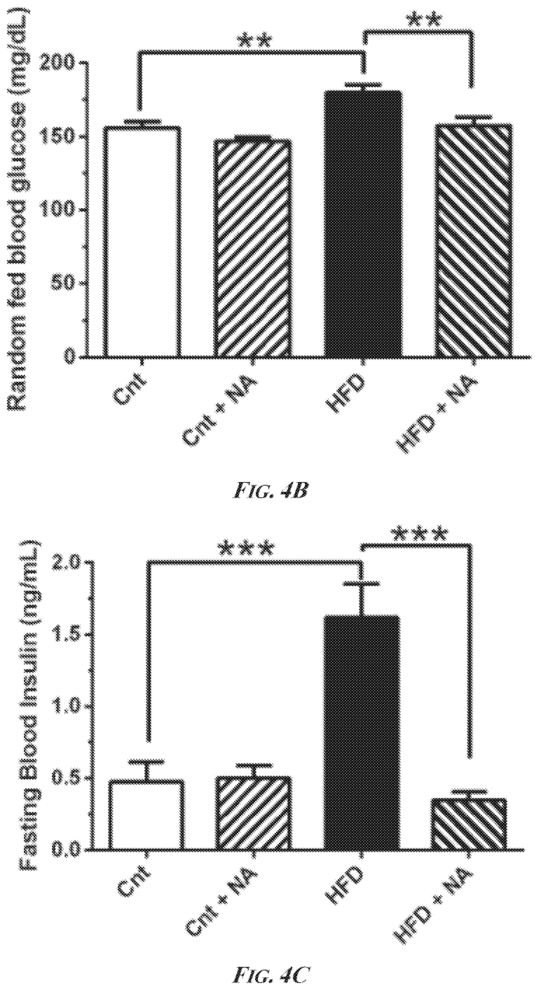

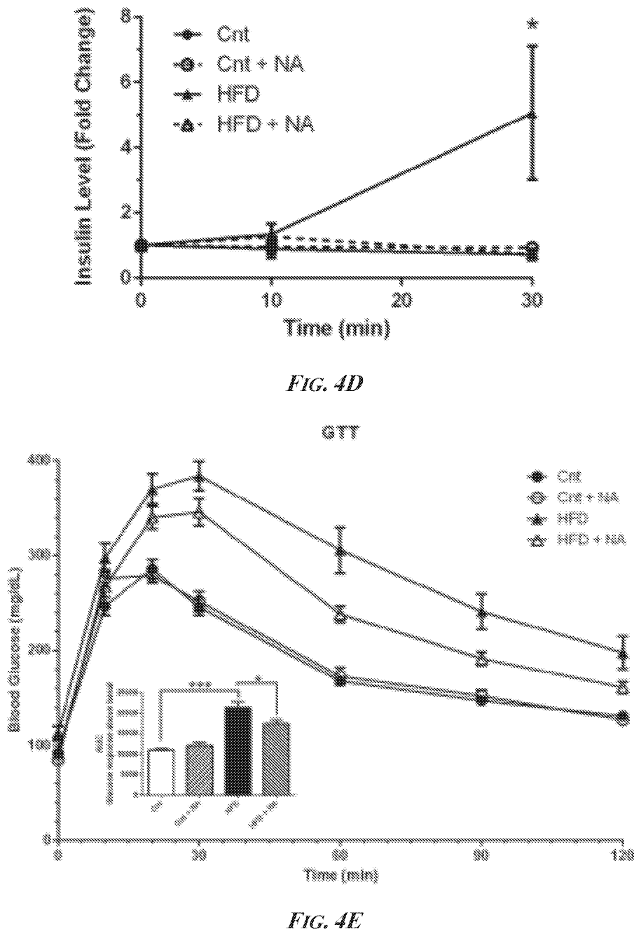

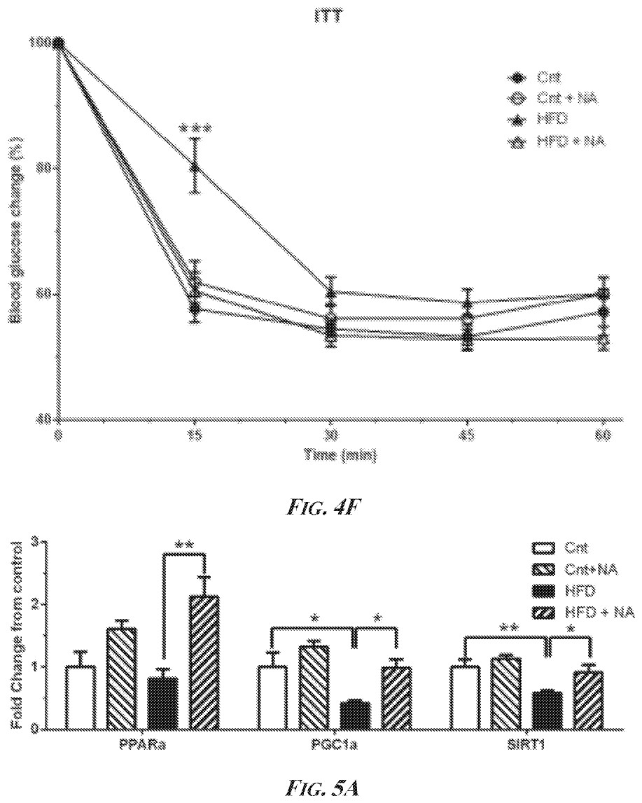

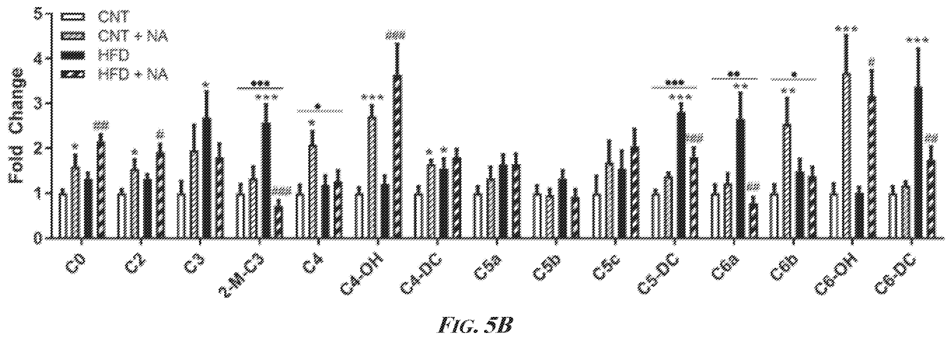

To determine metabolic consequences of dietary NA enrichment, circulating glucose and insulin levels under fasting and random-fed conditions were determined and glucose and insulin tolerance tests were performed. After 8-weeks, the HFD led to increased fasting (FIG. 4A) and random-fed (FIG. 4B) blood glucose levels when compared with control. Mice on HFD+NA demonstrated significant reductions in blood glucose levels compared to their HFD fed counterparts under both random-fed and fasted conditions. Blood glucose levels of mice on the HFD+NA diet were not significantly different from mice on control diets under random fed or fasted conditions. No significant differences were observed between mice on a control and mice on a control with NA diet.

Fasting plasma insulin levels were measured after 8 weeks on diets (FIG. 4C). In the control diet, NA did not affect insulin levels. Mice on the HFD demonstrated a significant increase in basal insulin levels, whereas in mice on the HFD+NA insulin levels, when compared to HFD mice, were significantly reduced to levels not significantly different from mice on control diets. Insulin levels were also assessed after intraperitoneal administration of glucose (FIG. 4D). Mice on the HFD showed a significant increase in insulin after 30 minutes, while mice on the HFD+NA showed no such increase. No discernable differences in insulin levels were observed at any of the time points analyzed in the control groups.

Glucose tolerance tests were performed after 10 weeks on diets (FIG. 4E). Impaired glucose tolerance was observed in mice on both high fat diets compared to control diets. However, mice on the HFD+NA showed significantly improved glucose tolerance over HFD. To assess insulin sensitivity, insulin tolerance tests were performed at 11 weeks on diets (FIG. 4F). Mice on the HFD showed a diminished response to lower blood glucose levels after insulin injection, whereas mice on the HFD+NA responded similarly to mice on control diets. Mice on the control diet containing NA responded similar to mice on the control diet alone. Taken together, the HFD enriched with NA normalized blood glucose and insulin levels, and improved glucose tolerance and insulin sensitivity when compared to the HFD alone.

### Example 6

### Nervonic Acid Improved Biomarkers of Energy Metabolism in the Liver

Markers of energy metabolism in the liver were next assayed by RT-PCR (see FIG. 5A. The HFD significantly reduced transcript levels of PGC1α and SIRT1, but not PPARα, while the HFD+NA increased mRNA levels of PPARα and restored PGC1α and SIRT1 back to control levels.

As these genes regulate the expression of genes involved in fatty acid β-oxidation and are major regulators of metabolism, acylcarnitine levels were analyzed as a functional readout of altered cellular metabolism by a liquid chromatography-mass spectrometry approach. The graphs of short-chain (FIG. 5B), medium-chain (FIG. 5C), and long-chain acylcarnines (FIG. 5D) depict alterations in hydroxyl- and non-hydroxyl-acylcarnitines species from C0-C20 due to the diets. The most marked differences observed between the control and HFD-fed mice were significant increases in many medium and long-chain (C10-C20) acylcarnitine species (see FIGS. 5C and 5D). These changes are consistent with impaired fatty acid oxidation and could be a marker of insulin resistance (see e.g., Reuter & Evans, 2012; Schooneman et al., 2013). In contrast, mice on a HFD+NA showed significant decreases in these long-chain acylcarnitines compared to the HFD. Furthermore, the NA enrichment, in both the control diet and the HFD, led to increases in free carnitine (C0), acetyl-carnitine (C2) and several hydroxyl-fatty acylcarnitines (C4-OH. C6-OH, and C10-OH; see FIGS. 5B and 5C). Increases in hydroxy-carnitines, an intermediate in fatty acid oxidation, may reflect increased higher lipid flux, with increases in C2-carnitine being an indication of elevated fatty acid oxidation.

Taken together, these results indicate that NA-enriched diets can increase fatty acid utilization via β-oxidation, possibly as a function of increased, or restored, PPARα/PGC1α/SIRT1 transcription.

### Discussion of the Examples

Disclosed herein is the demonstration that dietary NA supplementation provided protection against HFD-induced obesity and associated metabolic complications. NA and other very long chain fatty acid containing ceramides were reduced in the liver of mice and rats on a HFD (FIG. 1) and HFD+NA reversed these changes. Simultaneously, HFD+NA decreased body weight gains and reduced adiposity, while not changing food intake or absorption (FIG. 2). Mice on a HFD enriched with NA demonstrated energy expenditure and heat production similar to control mice but had similar RER and activity levels as HFD-fed counterparts (FIG. 3). Improved body composition with decreased adiposity in HFD+NA mice was associated with better glycemic control and increased insulin sensitivity (FIG. 4). Better energy utilization in HFD+NA mice was suggested by increased liver expression of PPARα, PGC1α, SIRT1, and improved fatty acid oxidation was supported by altered acylcarnitine profiles in mice on the NA-supplemented HFD (FIG. 5). This is the first evidence of a link between NA levels in liver and the development of obesity and related metabolic complications.

A few human studies support the observations presented herein with preclinical animal models that reduced NA could contribute to obesity and the metabolic syndrome. A negative correlation between circulating NA levels and obesity-related risk factors has been described in a previous study, and the authors concluded that NA might prevent obesity-related metabolic disorders (Oda et al., 2005). Another group demonstrated that plasma NA levels were significantly lower in obese compared to lean participants and were inversely correlated with BMI (Pickens et al., 2015). Furthermore, a study with Japanese males found that subjects with metabolic syndrome demonstrated reduced NA in serum lipids compared to subjects without metabolic syndrome (Yamazaki et al., 2014).

In contrast to these studies, another study reported that obese adolescent females, but not males, had increased NA (Karlsson et al., 2006), and in another study, obese young male adults did not exhibit changes in NA-containing sphingomyelin or ceramides (Hanamatsu et al., 2014). An additional study reported that a low-calorie diet reduced NA in erythrocyte membranes of overweight/obese persons who lost at least 5% of their initial body weight (Cazzola et al., 2011). The discrepancies between these studies are not clear, though age, gender, sample type, and the patient population could have affected results.

Investigations into saturated and monounsaturated very-long chain fatty acids (>22 carbons), such as NA, have been fairly limited. It is presently unknown if the effect of NA in mice on a HFD is specific or if it extends to other very long chain fatty acids, such as C22:0, C22:1 and C24:0. Supporting the presently disclosed subject matter, behenic acid (C22:0), in triglyceride form (1 molecule of C22 with 2 molecules of oleic acid), prevented obesity in rats via suppressed triglyceride absorption through pancreatic lipase inhibition (Kojima et al., 2010). Yet, alterations in fecal caloric content were not observed in the present experiments, so this mechanism might not apply to effects of the NA-enriched HFD used herein although it should be noted that bomb calorimetry does not differentiate between non-digestible and utilizable energy. No overt toxicities were observed in animals fed a NA-enriched diet (e.g., ruffled fur, anorexia, cachexia, skin tenting, skin ulcerations, diarrhea, or death). However, a closely related fatty acid, erucic acid (C22:1) in rapeseed oil was implicated in causing heart lesions in rats (Hulan et al., 1977), though another group suggested linolenic acid as the culprit (Dewailly et al., 1978). Correlative studies suggested ω-9 fatty acids, including NA, increase the risk of all-cause or cardiovascular mortality (Delgado et al., 2017). Interestingly, it has been further demonstrated that very-long chain ceramides exhibit a lipotoxic effect in cardiomyocytes (Law et al., 2018). Due to the controversies of ω-9 fatty acid supplementation, studies to investigate fatty acid specificity, dose responsiveness, fatty acid delivery formulations (free, ester, acylated), and toxicology of NA supplementation are still needed.

The beneficial effect of NA could be exerted through re-acylation of dietary NA into sphingolipids to restore C24:1-ceramide levels (see FIG. 1A). The data presented herein is consistent with this restoration occurring through ceramide remodeling as total amounts of ceramides were consistent between diet groups (see FIG. 1A, inset). The data presented herein also suggested that very-long chain fatty acids, such as NA, are selectively acylated into sphingolipids as opposed to glycerolipids (see FIGS. 1A-H; see also Christophersen et al., 1983; Fox et al., 2011). However, effects of other lipid classes or NA as a circulating free fatty acid could also be contributing factors.

The role of specific ceramide fatty acid composition, rather than total ceramide, has gained greater appreciation in disease states. C16-ceramide has been implicated in palmitate-induced insulin resistance and impaired fatty acid β-oxidation. C16-ceramides are significantly elevated in skeletal muscles and adipose tissues but not in livers of obese mice (see FIG. 1; see also Raichur et al., 2014; Gosejacob et al., 2016). Thus, effects could be tissue specific. Recently, it has been shown that the lipid enzyme CerS4, that generates C18 and C20-ceramides, contributes to hepatic insulin resistance (Matsuzaka et al., 2020). Consistently, in HFD-fed mice, increases in hepatic C18, C20 and C22 ceramides were observed and the NA-supplemented diet returned C20 and C22-ceramides back to control levels. Thus, indirectly, increasing NA could improve outcomes by decreasing detrimental effects of other ceramides in our mouse model. In vitro CerS2 knockdown data for C24:1-ceramide (see FIG. 1J) demonstrated a similar pattern to what is observed in liver upon NA addition (FIG. 1A). Data from knockout mice supports an apparent lack of functional redundancy for CerS2 in generating C24 containing sphingolipids (Imgrund et al., 2009; Pewzner-Jung et al., 2010) though reverse ceramidase activity (Okino et al., 2003) cannot be ruled out as a potential pathway for acylation in the present studies. Independent of fatty acid composition, it was also recently demonstrated that preventing the conversion of dihydroceramides to ceramides was sufficient to resolve hepatic steatosis and insulin resistance in obese mice (Chaurasia et al., 2019). Furthermore, deoxysphingolipids that are generated when alanine is used instead of serine in de novo sphingolipid synthesis were also implicated in contributing to diabetes-related complications (Othman et al., 2015; Mwinyi et al., 2017). Taken together, while long-chain fatty acid-derived ceramides have been implicated in detrimental effects of obesity and diabetes, the presently disclosed studies are the first to document that very-long chain C24:1 species can lead to opposite effects. A similar dichotomy of the physiological actions of individual ceramide species is now being appreciated in cardiovascular and oncological diseases (Grosch et al., 2012; Androedh et al., 2018).

Mechanistically, disclosed herein is the discovery that NA supplementation increased PPARα and restored PGC-1α and SIRT1 expression levels in mice on a HFD.

These targets have been implicated in obesity-related complications as they increase hepatic fatty acid oxidation and decrease the levels of circulating triglycerides responsible for adiposity (Yoon, 2009; Cheng et al., 2018). Evidence for increased fatty acid oxidation was observed when acylcarnitines were examined. Mice on a HFD demonstrated a significant increase in several long-chain acylcarnitines that are largely restored to control levels when the HFD is enriched with NA. This observation implicates that impaired fatty acid β-oxidation in mice on a HFD is ameliorated with NA supplementation, which could then limit the detrimental effects of long-chain acylcarnitines, including insulin resistance, detergent effect, lipotoxicity, and reduced glucose utilization (Reuter & Evans, 2012; Schooneman et al., 2013). Independent of diet, NA enrichment increased free carnitine, C2, C4-OH, C8-OH carnitines suggesting increased rates of hepatic fatty acid oxidation. Elevated C4-OH could also reflect increased ketogenesis (Hack et al., 2006) and has been implicated in insulin resistance (An et al., 2004). Hydroxyl- and dicarboxyl-fatty acids are also products of fatty acid ω-oxidation, which can be elevated with a HFD (Reddy & Rao, 2006; Hardwich et al., 2009). As disclosed herein, teductions of the dicarboxyl-acylcarnitines C5 (C5-DC) and C6 (C6-DC) levels in mice on a HFD enriched with NA and improved glucose/insulin tolerance were observed, suggesting that increased C4-OH predominantly reflects an intermediate in β-oxidation. With improved fatty acid oxidation, a likely consequence of upregulation of energy metabolism regulators, PPARα, PGC-1α, and SIRT1, the transcriptional mechanisms underlying this NA-induced upregulation is under active investigation.

Significant differences in whole body fatty acid oxidation (RER) or parameters of whole-body energy homeostasis that could explain the substantial decrease in body weight with HFD+NUCLEIC ACID were not observed. However, studies into energy expenditure are inherently underpowered (Tschop et al., 2011). The presently disclosed studies have about 80% power to detect a medium effect size. Consequently, small changes in for example energy expenditure and food intake that could have been missed could still have a profound cumulative effect on body weight.

Summarily, as disclosed herein, a HFD specifically reduced very-long chain sphingolipids, in particular the abundant C24:1 nervonic acid, and that restoring NA levels in mice on HFD reduced body weight gain and improved metabolic parameters. Although more investigation is needed into the mechanisms by which NA supplementation exerts effects, the presently disclosed subject matter supports the clinical relevance of treatment of metabolic disorders. Increasing dietary NA to restore HFD-induced reduction of long-chain fatty acids could be an effective way to improve the management of obesity and associated metabolic complications including diabetes.

### REFERENCES

All references listed in the instant disclosure and in the Appendices attached hereto, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Altschul et al. (1990) Basic local alignment search tool. Journal of Molecular Biology 215:3:403-410.
Altschul et al. (1997) Gapped BLAST and PSI-BLAST: a new generation of protein database search programs. Nucleic Acids Res 25:3389-3402.
An et al. (2004) Hepatic expression of malonyl-CoA decarboxylase reverses muscle, liver and whole-animal insulin resistance. Nature Medicine 10:268-274.
Anroedh et al. (2018) Plasma concentrations of molecular lipid species predict long-term clinical outcome in coronary artery disease patients. Journal of Lipid Research 59:1729-1737.
Astete et al. (2006) Synthesis and characterization of PLGA nanoparticles. J Biomater Sci Polymer Edn 17:247-289.
Bandet et al. (2019) Sphingolipid Metabolism: New Insight into Ceramide-Induced Lipotoxicity in Muscle Cells. International Journal of Molecular Sciences 20:479.
Betageri et al. (1993) Liposome Drug Delivery Systems, Technomic Publishing, Lancaster, Pa., United States of America.
Calhoun et al. (1997) Treatment of osteomyelitis with a biodegradable antibiotic implant. Clin Orthopaed Related Res 341:206-214.
Cazzola et al. (2011) Effects of weight loss on erythrocyte membrane composition and fluidity in overweight and moderately obese women. The Journal of Nutritional Biochemistry 22:388-392.
Chaurasia et al. (2019) Targeting a ceramide double bond improves insulin resistance and hepatic steatosis. Science 365:386-392.
Cheng et al. (2018) PGC-1α as a Pivotal Factor in Lipid and Metabolic Regulation. International Journal of Molecular Sciences 19:3447.
Christophersen et al. (1983) Metabolism of erucic acid in adipocytes isolated from rat epididymal fat. Lipids 18:137-141.
de Faria et al. (2005) Preparation and Characterization of Poly(D,L-Lactide) (PLA) and Poly(D,L-Lactide)-Poly(Ethylene Glycol) (PLA-PEG) Nanocapsules Containing Antitumoral Agent Methotrexate. Macromol Symp 229:228-233.
Delgado et al. (2017) Individual omega-9 monounsaturated fatty acids and mortality—The Ludwigshafen Risk and Cardiovascular Health Study. Journal of Clinical Lipidology 11:126-135 e125.
Devereux et al. (1984) A comprehensive set of sequence analysis programs for the VAX. Nucl Acids Res 12:387.
Dewailly et al. (1978) Changes in fatty acid composition of cardiac mitochondrial phospholipids in rats fed rapeseed oil. Lipids 13:301-304.
Fox & Kester (2010) Therapeutic strategies for diabetes and complications: a role for sphingolipids? Advances in Experimental Medicine and Biology 688:206-216.
Fox et al. (2011) Circulating sphingolipid biomarkers in models of type 1 diabetes. Journal of Lipid Research 52:509-517.
Gerhardt et al. (eds.) (1994) Methods for General and Molecular Bacteriology, American Society for Microbiology, Washington, D.C., page 574.
Giesbertz et al. (2015) An LC-MS/MS method to quantify acylcarnitine species including isomeric and odd-numbered forms in plasma and tissues. Journal of Lipid Research 56:2029-2039.
Gonsalves et al. (1998) Synthesis and surface characterization of functionalized polylactide copolymer microparticles. Biomaterials 19:1501-1505.
Gosejacob et al. (2016) Ceramide Synthase 5 Is Essential to Maintain C16:0-Ceramide Pools and Contributes to the Development of Diet-induced Obesity. The Journal of Biological Chemistry 291:6989-7003.
Gregoriadis, ed. (1993) Liposome Technology, CRC Press, Boca Raton, Fla., United States of America.
Grosch et al. (2012) Chain length-specific properties of ceramides. Progress in Lipid Research 51:50-62.
Hack et al. (2006) Monitoring of ketogenic diet for carnitine metabolites by subcutaneous microdialysis. Pediatric Research 60:93-96.
Hammad & Jones (2017) Dietary Fatty Acid Composition Modulates Obesity and Interacts with Obesity-Related Genes. Lipids 52:803-822.
Hanamatsu et al. (2014) Altered levels of serum sphingomyelin and ceramide containing distinct acyl chains in young obese adults. Nutrition & Diabetes 4:e141.
Hans et al. (2002) Synthesis and characterization of mPEG-PLA prodrug micelles. Solid State Mater Sci 6:319-327.
Hardwick et al. (2009) PPAR/RXR Regulation of Fatty Acid Metabolism and Fatty Acid omega-Hydroxylase (CYP4) Isozymes: Implications for Prevention of Lipotoxicity in Fatty Liver Disease. PPAR Research 2009:952734.
Hulan et al. (1977) Myocardial lesions in rats fed rapeseed oil. I. Influence of strain of rats. Canadian Journal of Physiology and Pharmacology 55:258-264.
Imgrund et al. (2009) Adult ceramide synthase 2 (CERS2)-deficient mice exhibit myelin sheath defects, cerebellar degeneration, and hepatocarcinomas. The Journal of Biological Chemistry 284:33549-33560.
Janoff, ed. (1999) Liposomes: Rational Design, M. Dekker, New York, N.Y., United States of America.
Karlin & Altschul (1990) Methods for assessing the statistical significance of molecular sequence features by using general scoring schemes. Proceedings of the National Academy of Sciences of the United States of America 87:2264-2268.
Karlin & Altschul (1993) Applications and statistics for multiple high-scoring segments in molecular sequences. Proceedings of the National Academy of Sciences of the United States of America 90:14:5509-5013.
Karlsson et al. (2006) Serum phospholipid fatty acids, adipose tissue, and metabolic markers in obese adolescents. Obesity 14:1931-1939.
Kojima et al. (2010) Structured triacylglycerol containing behenic and oleic acids suppresses triacylglycerol absorption and prevents obesity in rats. Lipids in Health and Disease 9: 77.
Kumar et al. (2004) Preparation and characterization of cationic PLGA nanospheres as DNA carriers. Biomaterials 25:1771-1777.
Kwon et al. (2001) Preparation of PLGA nanoparticles containing estrogen by emulsification-diffusion method. Colloids Surf A 182:123-130).
Labat-Moleur et al. (1996) An electron microscopy study into the mechanism of gene transfer with lipopolyamines. Gene Therapy 3:1010-1017.
Lambert et al. (2018) Sphingolipids in adipose tissue: What's tipping the scale? Advances in Biological Regulation 70:19-30.
Lamprecht et al. (1999) Biodegradable monodispersed nanoparticles prepared by pressure homogenization-emulsification. Intl J Pharmaceut 184:97-105.
Lansey et al. (2012) Deletion of Rab GAP AS160 modifies glucose uptake and GLUT4 translocation in primary skeletal muscles and adipocytes and impairs glucose homeostasis. American Journal of Physiology. Endocrinology and Metabolism 303:E1273-1286.
Lasic & Martin (1995) Stealth Liposomes, CRC Press, Boca Raton, Fla., United States of America.
Laurencin et al. (2001) Poly(lactide-co-glycolide)/hydroxyapatite delivery of BMP-2-producing cells: a regional gene therapy approach to bone regeneration. Biomaterials 22:1271-1277.
Law et al. (2018) Lipotoxic very-long-chain ceramides cause mitochondrial dysfunction, oxidative stress, and cell death in cardiomyocytes. FASEB Journal 32:1403-1416.
Mandal et al. (2002) Poly(D,L-lactide-co-glycolide) encapsulated poly(vinyl alcohol) hydrogel as a drug delivery system. Pharmaceut Res 19:1713-1719.
Matsuzaka et al. (2020) Hepatocyte ELOVL Fatty Acid Elongase 6 Determines Ceramide Acyl-Chain Length and Hepatic Insulin Sensitivity in Mice. Hepatology 71:1609-1625.
Meikle & Summers (2017) Sphingolipids and phospholipids in insulin resistance and related metabolic disorders. Nature Reviews Endocrinology 13:79-91.
Mwinyi et al. (2017) Plasma 1-deoxysphingolipids are early predictors of incident type 2 diabetes mellitus. PloS one 12:e0175776.
Oda et al. (2005) Relationships between serum unsaturated fatty acids and coronary risk factors: negative relations between nervonic acid and obesity-related risk factors. International Heart Journal 46:975-985.
Okino et al. (2003) The reverse activity of human acid ceramidase. The Journal of Biological Chemistry 278″29948-29953.
Othman et al. (2015) Lowering plasma 1-deoxysphingolipids improves neuropathy in diabetic rats. Diabetes 64:1035-1045.
Palomer et al. (2018) Palmitic and Oleic Acid: The Yin and Yang of Fatty Acids in Type 2 Diabetes Mellitus. Trends in Endocrinology and Metabolism 29:178-190.
Park et al. (2014) Ceramide synthases as potential targets for therapeutic intervention in human diseases. Biochimica et Biophysica Acta 1841:671-681.
Pearson et al. (2020) Ceramide Analogue SACLAC Modulates Sphingolipid Levels and MCL-1 Splicing to Induce Apoptosis in Acute Myeloid Leukemia. Molecular Cancer Research 18:352-363.
Pewzner-Jung et al. (2010) A critical role for ceramide synthase 2 in liver homeostasis: I. alterations in lipid metabolic pathways. The Journal of Biological Chemistry 285:10902-10910.
Pickens et al. (2015) Plasma phospholipids, non-esterified plasma polyunsaturated fatty acids and oxylipids are associated with BMI. Prostaglandins, Leukotrienes, and Essential Fatty Acids 95:31-40.
Raichur et al. (2014) CerS2 haploinsufficiency inhibits beta-oxidation and confers susceptibility to diet-induced steatohepatitis and insulin resistance. Cell Metabolism 20:687-695.
Reddy & Rao (2006) Lipid metabolism and liver inflammation. II. Fatty liver disease and fatty acid oxidation. American Journal of Physiology. Gastrointestinal and Liver Physiology 290:G852-858.
Reuter & Evans (2012) Carnitine and acylcarnitines: pharmacokinetic, pharmacological and clinical aspects. Clinical Pharmacokinetics 51:553-572.
Schooneman et al. (2013) Acylcarnitines: reflecting or inflicting insulin resistance?Diabetes 62: 1-8.
Song et al. (1997) Formulation and characterization of biodegradable nanoparticles for intravascular local drug delivery. J Controlled Release 43:197-212.
Soppimath et al., (2001) Biodegradable polymeric nanoparticles as drug delivery devices. J Controlled Release 70:1-20.
Tschop et al. (2011) A guide to analysis of mouse energy metabolism. Nature Methods 9:57-63.
Turpin et al. (2014) Obesity-induced CerS6-dependent C16:0 ceramide production promotes weight gain and glucose intolerance. Cell Metabolism 20:678-686.
U.S. Patent Application Publication Nos. 2011/0104069, 2013/0330279, 2018/0078657, 2019/0091280, 2020/0038452.
U.S. Pat. Nos. 4,235,871; 4,551,482; 5,011,634; 5,948,767; 6,056,938; 6,132,766; 6,180,082; 6,197,333; 6,200,598; 6,210,707; 6,217,886; 7,332,586; 7,901,711; 8,137,697; 8,449,915; 8,663,599.
Wijesinghe et al. (2010) Use of high performance liquid chromatography-electrospray ionization-tandem mass spectrometry for the analysis of ceramide-1-phosphate levels. Journal of Lipid Research 51:641-651.
Yamazaki et al. (2014) Proportion of nervonic acid in serum lipids is associated with serum plasmalogen levels and metabolic syndrome. Journal of Oleo Science 63:527-537.
Yoon (2009) The role of PPARα in lipid metabolism and obesity: focusing on the effects of estrogen on PPARα actions. Pharmacological Research 60:151-159.

It will be understood that various details of the presently disclosed subject matter can be changed without departing from the scope of the presently disclosed subject matter. Furthermore, the foregoing description is for the purpose of illustration only, and not for the purpose of limitation.

1. A method for preventing and/or reducing weight gain in a subject, optionally a mammal, further optionally a human, the method comprising, consisting essentially of, or consisting of administering to the subject an effective amount of a composition comprising, consisting essentially of, or consisting of long chain fatty acid of at least 22 carbons and/or a derivative thereof, optionally wherein the long chain fatty acid is a monounsaturated omega 9 fatty acid, wherein the effective amount of the composition is effective for preventing and/or reducing weight gain in the subject relative to that which would have occurred in the subject in the absence of the composition.

2. A method for preventing and/or reducing the development of obesity in a subject, optionally a mammal, further optionally a human, the method comprising, consisting essentially of, or consisting of administering to the subject an effective amount of a comprising, consisting essentially of, or consisting of long chain fatty acid of at least 22 carbons and/or a derivative thereof, optionally wherein the long chain fatty acid is a monounsaturated omega 9 fatty acid.

3. The method of claim 1, wherein the long chain fatty acid is selected from the group consisting of erucic acid, nervonic acid, and ximenic acid.

4. The method of claim 1, wherein the derivative of the of long chain fatty acid of at least 22 carbons is a precursor thereof, optionally a metabolic precursor thereof, a metabolite thereof, an analog thereof, an ester thereof, an ether thereof, an amide thereof, a pharmaceutically acceptable salt thereof, or any combination thereof,

5. The method of claim 1, wherein the derivative comprises the long chain fatty acid bioconjugated to a biomolecule selected from the group consisting of a ceramide, a lipid, a phospholipid, a cholesteroal, a dyglyceride, a triglyceride, a monoacylglycerol, and a glycerophopholipid.

6. The method of claim 1, wherein the long chain fatty acid if bioconjugated to the biomolecule via an ester linkage, an ether linkage, an amide linkage, or any combination thereof.

7. The method of claim 1, wherein the derivative is an ester of the long chain fatty acid and a C₁-C₆ straight chain biomolecule, a C₁-C₆ branched chain biomoecule, or is any combination thereof.

8. The method of claim 7, wherein the derivative of the long chain fatty acid is a methyl ester, an ethyl ester, or any combination thereof.

9. The method of claim 1, wherein the composition comprises, consists essentially of, or consists of nervonic acid, optionally an ester thereof, and further optionally a methyl ester and/or an ethyl ester thereof.

10. The method of claim 1, wherein the composition comprises, consists essentially of, or consists of nervonic acid and/or a derivative thereof bioconjugated to a ceramide, optionally wherein the derivative thereof is a nervonic acid ethyl ester.

11. A method for inhibiting reduction of very-long chain sphingolipids in a subject, optionally a mammal, further optionally a human, the method comprising, consisting essentially of, or consisting of administering to the subject an effective amount of a composition comprising, consisting essentially of, or consisting of long chain fatty acid of at least 22 carbons, a precursor thereof, a metabolite thereof, an analog thereof, an ester thereof, an ether thereof, an amide thereof, a pharmaceutically acceptable salt thereof, or any combination thereof, optionally wherein the long chain fatty acid is a monounsaturated omega 9 fatty acid.

12. The method of claim 11, wherein the long chain sphingolipid is C24:1 nervonic acid.

13. The method of claim 11, wherein the long chain fatty acid is selected from the group consisting of erucic acid, nervonic acid, and ximenic acid.

14. The method of claim 11, wherein the ester thereof is a C₁-C₆ straight chain ester, C₁-C₆ branched chain ester, or any combination thereof.

15. The method of claim 11, wherein the ester thereof is a methyl ester, an ethyl ester, or any combination thereof.

16. The method of claim 11, wherein the composition comprises, consists essentially of, or consists of nervonic acid, optionally an ester thereof, and further optionally a methyl ester and/or an ethyl ester thereof.

17. The method of claim 11, wherein the weight gain, the obesity, or the reduction in very-long chain sphingolipids is a consequence of the subject consuming a high fat diet.

18. The method of claim 1, wherein the subject is a human.

19. A method for increasing content of one or more first species of ceramides in a subject, optionally a mammal, further optionally a human, while simultaneously decreasing content of one or more second species of C20-C26 ceramides in the subject, the method comprising, consisting essentially of, or consisting of administering to the subject an effective amount of a composition comprising, consisting essentially of, or consisting of an effective amount of a composition comprising, consisting essentially of, or consisting of long chain fatty acid of at least 22 carbons, a precursor thereof, a metabolite thereof, an analog thereof, an ester thereof, an ether thereof, an amide thereof, a pharmaceutically acceptable salt thereof, or any combination thereof, optionally wherein the long chain fatty acid is a monounsaturated omega 9 fatty acid, and further wherein the effective amount is effective for increasing content of the one or more first species of ceramides in the subject while simultaneously decreasing content of the one or more second species of C20-C26 ceramides in the subject.

20. The method of claim 19, wherein the one or more first species of ceramides increased are selected from the group consisting of C24:1-lysophosphatidylcholine an diacylglycerides C16:0/C24:1, C18:0/C24:1, C18:1/C24:1, and C18:2/C24:1.

21. The method of claim 19, wherein the content is plasma content, liver content, or both.

23. A method for reducing a blood glucose level resulting from consumption of a high fat diet in a subject in need thereof, the method comprising, consisting essentially of, or consisting of administering to the subject an effective amount of a composition comprising, consisting essentially of, or consisting of an effective amount of a composition comprising, consisting essentially of, or consisting of long chain fatty acid of at least 22 carbons, a precursor thereof, a metabolite thereof, an analog thereof, an ester thereof, an ether thereof, an amide thereof, a pharmaceutically acceptable salt thereof, or any combination thereof, optionally wherein the long chain fatty acid is a monounsaturated omega 9 fatty acid, wherein the effective amount is effective for reducing the blood glucose level in the subject.

24. The method of claim 23, wherein the long chain fatty acid is selected from the group consisting of erucic acid, nervonic acid, and ximenic acid.

25. The method of claim 23, wherein the ester thereof is a C₁-C₆ straight chain ester, C₁-C₆ branched chain ester, or any combination thereof.

26. The method of claim 23, wherein the ester thereof is a methyl ester, an ethyl ester, or any combination thereof.

27. The method of claim 23, wherein the composition comprises, consists essentially of, or consists of nervonic acid, optionally an ester thereof, and further optionally a methyl ester and/or an ethyl ester thereof.

28. The method of claim 1, wherein the long chain fatty acid is nervonic acid and the nervonic acid is selected from the group consisting of free nervonic acid, nervonic acid methyl ester, nervonic acid ethyl ester, and nervonic acid acylated to triglyceride or sphingolipid.

29. The method of claim 1, wherein the composition is administered as part of a nanoscale or microscale delivery vehicle, wherein the delivery vehicle is optionally selected from the group consisting of a liposome, a lipo/polymer, a microparticle, and a nanoparticle, or any combination thereof.

30. The method of claim 29, wherein the delivery vehicle comprises a nanoliposome, and further wherein the nanoliposome encompasses the long chain fatty acid, the precursor thereof, the metabolite thereof, the analog thereof, the ester thereof, the pharmaceutically acceptable salt thereof, the combination thereof and/or comprises a lipid bilayer that comprises the long chain fatty acid, the precursor thereof, the metabolite thereof, the analog thereof, the ester thereof, the pharmaceutically acceptable salt thereof, the combination thereof.

31. The method of claim 29, wherein the delivery vehicle is designed to degrade in the subject in order to release the long chain fatty acid of at least 22 carbons, the precursor thereof, the metabolite thereof, the analog thereof, the ester thereof, the pharmaceutically acceptable salt thereof, or any combination thereof to the subject over a period of time.

32. The method of claim 31, wherein the delivery vehicle releases the long chain fatty acid of at least 22 carbons, the precursor thereof, the metabolite thereof, the analog thereof, the ester thereof, the pharmaceutically acceptable salt thereof, or any combination thereof to the subject's circulation and/or a cell, tissue, and/or organ of subject over the period of time.

33. The method of claim 31, wherein the delivery vehicle is designed to degrade subsequent to contact with the subject's digestive system or circulatory system.

34. The method of claim 31, wherein the delivery vehicle is designed to degrade in the subject to release at least about 50% of the long chain fatty acid of at least 22 carbons, the precursor thereof, the metabolite thereof, the analog thereof, the ester thereof, the pharmaceutically acceptable salt thereof, or any combination thereof over a period of time of at least 30 minutes, at least 1 hour, at least 6 hours, at least 12 hours, at least 24 hours, or longer than 24 hours.
